(19) United States
(12) Patent Application Publication
(10) Pub. No.: US 2003/0224438 A1
(43) Pub. Date: Dec. 4, 2003

(54) Novel molecules of the PYRIN/NBS/LRR protein family and uses thereof

(75) Inventor: John Bertin, Toronto (CA)

Correspondence Address: Jean M. Silveri, Millennium Pharmaceuticals, Inc., 75 Sidney Street, Cambridge, MA, 02139, US

(73) Assignee: Millennium Pharmaceuticals, Inc.

(21) Appl. No.: 10/446,046

(22) Filed: May 23, 2003

Related U.S. Application Data
(60) Provisional application No. 60/383,487, filed on May 24, 2002.

(51) Int. Cl. C12Q 1/68; C07H 21/04; C12N 9/99; C12P 21/02; C12N 5/06

(52) U.S. Cl. 435/6; 435/69.2; 435/184; 435/320.1; 435/325; 536/23.2

(57) ABSTRACT

Novel PYRIN-9 polypeptides, proteins, and nucleic acid molecules are disclosed. In addition to isolated PYRIN-9 proteins, the invention further provides PYRIN-9 fusion proteins, antigenic peptides and anti-PYRIN-9 antibodies. The invention also provides PYRIN-9 nucleic acid molecules, recombinant expression vectors containing a nucleic acid molecule of the invention, host cells into which the expression vectors have been introduced and non-human transgenic animals in which a PYRIN-9 gene has been introduced or disrupted. Diagnostic, screening and therapeutic methods utilizing compositions of the invention are also provided.

### CROSS-REFERENCES TO RELATED APPLICATIONS

This application claims the benefit of U.S. Provisional Application No. 60/383,487, filed May 24, 2002, the contents of which are incorporated herein in their entirety by this reference.

### BACKGROUND OF THE INVENTION

Many cytoplasmic plant proteins involved in plant resistance to pathogens, generally referred to as “R” proteins, possess both a nucleotide binding site (NBS) and a leucine rich repeat (LRR). R proteins are involved in both a rapid defense response (hypersensitive response) and more long-term nonspecific resistance (systemic acquired resistance). The hypersensitive response involves a form of programmed death localized to the site of infection and changes in gene expression that are thought to prevent further infection. The LRR of the R proteins is believed to recognize and bind to pathogen-derived proteins, triggering the defensive responses and resulting in a rapid and localized host cell death. Many R proteins have an amino terminal effector domain (e.g., a TIR domain or a leucine zipper domain) that is thought to play a role in downstream signaling of events triggered by infection and, possibly, other stresses.

The R proteins are structurally similar to APAF-1, which mediates the activation of caspases, the proteases directly responsible for the degradation of cellular proteins that leads to the morphological changes seen in cells undergoing apoptosis. A domain, designated the NB-ARC domain (“nucleotide-binding adaptor shared by APAF-1, certain R gene products and CED-4”), contains a series of motifs and residues that are conserved among plant resistance proteins (e.g., R proteins) and regulators of cell death (e.g., APAF-1 and CED-4) (van der Bizen and Jones (1999) Current Biology 8:226-228). In addition to the NBS, APAF-1 has a CARD domain, functionally analogous to the effector domain of R proteins, and a WD-40 domain, functionally analogous to the LRR domain of R proteins.

The mechanisms that mediate apoptosis have been intensively studied. These mechanisms involve the activation of endogenous proteases, loss of mitochondrial function, and structural changes such as disruption of the cytoskeleton, cell shrinkage, membrane blebbing, and nuclear condensation due to degradation of DNA.

The various signals that trigger apoptosis are thought to bring about these events by converging on a common cell death pathway, the core components of which are highly conserved from worms, such as C. elegans, to humans. In fact, invertebrate model systems have been invaluable tools in identifying and characterizing the genes that control apoptosis. Despite this conservation of certain core components, apoptotic signaling in mammals is much more complex than in invertebrates. For example, in mammals there are multiple homologues of the core components in the cell death signaling pathway.

Caspases, a class of proteins central to the apoptotic program, are responsible for the degradation of cellular proteins that leads to the morphological changes seen in cells undergoing apoptosis. Caspases (cysteinyl aspartate-specific proteinases) are cysteine proteases having specificity for aspartate at the substrate cleavage site. Generally, caspases are classified as either initiator caspases or effector caspases, both of which are zymogens that are activated by proteolysis that generates an active species. An effector caspase is activated by an initiator caspase which cleaves the effector caspase. Initiator caspases are activated by an autoproteolytic mechanism that is often dependent upon oligomerization directed by association of the caspase with an adapter molecule.

Nuclear factor-κB (NF-κB) is a transcription factor expressed in many cell types and which activates homologous or heterologous genes that have κB sites in their promoters. Molecules that regulate NF-κB activation play a critical role in both apoptosis and inflammation. Quiescent NF-κB resides in the cytoplasm as a heterodimer of proteins referred to as p50 and p65 and is complexed with the regulatory protein IκB. NF-κB binding to IκB causes NF-κB to remain in the cytoplasm. At least two dozen stimuli that activate NF-κB are known (New England Journal of Medicine 336:1066, 1997) and they include cytokines, protein kinase C activators, oxidants, viruses, and immune system stimuli. NF-κB activating stimuli activate specific IκB kinases that phosphorylate IκB leading to its degradation. Once liberated from IκB, NF-κB translocates to the nucleus and activates genes with κB sites in their promoters. The proinflammatory cytokines TNF-α and IL-1 induce NF-κB activation by binding their cell-surface receptors and activating the NF-κB-inducing kinase, NIK, and NF-κB. NIK phosphorylates the IκB kinases α and β which phosphorylate IκB, leading to its degradation.

NF-κB and the NF-κB pathway has been implicated in mediating chronic inflammation in inflammatory diseases such as asthma, ulcerative colitis, rheumatoid arthritis (Epstein, New England Journal of Medicine 336:1066, 1997) and inhibiting NF-κB or NF-PB pathways may be an effective way of treating these diseases. NF-κB and the NF-κB pathway has also been implicated in atherosclerosis (Navab et al., American Journal of Cardiology 76:18C, 1995), especially in mediating fatty streak formation, and inhibiting NF-κB or NF-κB pathways may be an effective therapy for atherosclerosis. Among the genes activated by NF-κB are cIAP-1, cIAP-2, TRAF1, and TRAF2, all of which have been shown to protect cells from TNF-α induced cell death (Wang et al., Science 281:1680-83, 1998). CLAP, a protein which includes a CARD, activates the Apaf-1-caspase-9 pathway and activates NF-κB by acting upstream of NIK and IκB kinase (Srinivasula et al., supra).

CARD-4 is a member of the CED-4/Apaf-1 family that interacts with RICK, a serine threonine kinase, and induces NF-κB via the signaling protein TRAF-6 and NIK (Bertin et al. (1999) J. Biol. Chem. 274:12955). CARD-4 includes domains that are similar to the nucleotide binding site domain (NBS) and leucine rich repeat (LRR) domains found in plant R proteins that mediate resistance to pathogens.

CARD-4 and NOD2 (CARD-15) each activate NF-κB following assembly with RICK, a CARD-containing kinase that participates in both innate and adaptive immune responses (Kobayashi et al. (2002) Nature 416:194-99; Chin et al. (2002) Nature 416:190-94). Confirming the importance of NOD2 in inflammatory signaling, mutations within its gene confer susceptibility to Crohn's disease and Blau syndrome, two chronic immune disorders (Hugot et al. (2001) Nature 411:537-39; Ogura et al. (2001) Nature 411:603-06; Miceli-Richard et al. (2001) Nat. Genet. 29:19-20). CARD-12 also plays a role in inflammatory signaling by binding to and activating caspase-1, a CARD-containing caspase that processes pro-interleukin-1β and pro-interleukin-18 into biologically active cytokines.

### SUMMARY OF THE INVENTION

The invention features human PYRIN-9 polypeptides and nucleic acid molecules encoding human PYRIN-9. PYRIN-9 has a pyrin domain, so-named for its homology to a portion of pyrin (marenostrin). Mutations in the pyrin gene are associated with familial Mediterranean fever (FMF), an inherited inflammatory disease. PYRIN-9 also has a nucleotide binding site (NBS) domain, which is present in a number of proteins that transmit signals which activate apoptotic and inflammatory pathways in response to stress and other stimuli. PYRIN-9 also has a leucine rich repeat domain (LRR) domain, another domain present in a number of proteins involved in apoptotic and inflammatory pathways.

The invention is based, at least on part, on the discovery that PYRIN-9 bears homology to members of the PYPAF (PYRIN-containing Apaf1-like) family of proteins (see, e.g., Bertin et al. (2000) Cell Death Differ. 7:1273-74), PYRIN-9 co-localizes with CARD-5 in a cell, PYRIN-9 is expressed in peripheral blood leukocytes, PYRIN-9 and CARD-5 synergistically activate NF-κB, and PYRIN-9 and CARD-5 synergistically activate caspase-1.

The expression of PYRIN-9 in peripheral blood leukocytes indicates a role for the protein in inflammatory signaling. Both pro-IL-1β and pro-IL-18 require cleavage by caspase-1 to generate active cytokines (Fantuzzi et al. (1999) J. Clin. Immunol. 19:1-11). PYRIN-9 may therefore play an important role in innate and adaptive immunity by regulating the production of IL-1 and IL-18 by immune cells. CARD-5 (also known as ASC) has been identified as a signaling partner of CARD-7 and pyrin. CARD-7 and pyrin may regulate the activation of NF-κB and pro-caspase-1 through binding to CARD-5. CARD-5 may therefore function as an adaptor protein that links pyrin domain-containing proteins to downstream signaling pathways. The experimental results presented herein demonstrate a role for PYRIN-9 in the transduction of pro-inflammatory signals via the activation of CARD-5 and pro-caspase-1.

PYRIN-9 nucleic acids and polypeptides, as well as modulators of PYRIN-9 activity or expression, are expected to be useful in the modulation of stress-related, apoptotic and inflammatory responses, e.g., for the treatment of apoptotic and inflammatory disorders. In addition, PYRIN-9 nucleic acids and polypeptides are expected to be useful in the diagnosis of apoptotic and inflammatory disorders as well as in screening assays which can be used to identify compounds which can be used to modulate stress-related, apoptotic and inflammatory responses.

NBS-1, NBS-2, NBS-3, PYRIN-12/NBS-4, NBS-5, PYRIN-1, PYRIN-2, PYRIN-3, PYRIN-5, PYRIN-6, PYRIN-7, PYRIN-8, PYRIN-10, and PYRIN-11 have a pyrin domain, a NBS domain, and/or an LRR domain. As described herein, the pyrin domain is an effector domain thought to be involved in homophilic protein-protein interactions. The pyrin domain typically can be found at or near the N-terminus of the protein containing the pyrin domain. Detailed information concerning NBS-1, NBS-2, NBS-3, PYRIN-12/NBS-4, NBS-5, PYRIN-1, PYRIN-2, PYRIN-3, PYRIN-5, PYRIN-6, PYRIN-7, PYRIN-8, PYRIN-10, and PYRIN-11 can be found in U.S. application Ser. No. 09/506,067, filed Feb. 17, 2000, U.S. application Ser. No. 09/506,067, filed Sep. 1, 2000, U.S. application Ser. No. 09/848,035, filed May 3, 2001, U.S. application Ser. No. 10/124,498, filed Apr. 17, 2002, and U.S. application Ser. No. 10/127,516, filed Apr. 22, 2002, all of which are incorporated herein by reference.

CARD-4, CARD-7, and CARD-12 have both an NBS domain and an LRR domain as well as a CARD domain (detailed information concerning CARD-4, CARD-7, and CARD-12 can be found in U.S. application Ser. No. 09/245,281, filed Feb. 5, 1999, now U.S. Pat. No. 6,369,196, U.S. application Ser. No. 09/207,359, filed Dec. 8, 1998, now U.S. Pat. No. 6,469,140, U.S. application Ser. No. 09/099,041, filed Jun. 17, 1998, now U.S. Pat. No. 6,340,576, U.S. application Ser. No. 09/019,942, filed Feb. 6, 1998, now U.S. Pat. No. 6,033,855, U.S. application Ser. No. 09/428,252, filed Oct. 27, 1999, U.S. application Ser. No. 09/697,089, filed Oct. 26, 2000, and U.S. application Ser. No. 09/841,739, filed Apr. 24, 2001, all of which are incorporated herein by reference). The CARD domain, which is present in a number of apoptotic signaling molecules, is an effector domain that is thought to be involved in homophilic protein-protein interactions, e.g., with downstream CARD-containing signaling molecules. For example, the CARD domain of CARD-4 interacts with the CARD domain of RICK (RIP2, CARDIAK), a serine-threonine kinase that activates NF-κB signaling pathways.

In general, an NBS domain includes a kinase 1a domain (P-loop), a kinase 2 domain (Walker B box) and a kinase 3a domain. PYRIN-9 are believed to belong to the NACHT (AIP, CIIA, ET-E and TP1) subfamily of NBS-domain containing proteins. Members of the NACHT subfamily contain additional motifs common among subfamily members (see, e.g., Koonin et al. (2000) Trends Biochem. Sci. 25:223). NACHT NTPase subfamily members have been implicated in apoptosis and MHC transcription activation. Other members of the NACHT NTPase subfamily include CARD-4, CARD-7, NAIP, NBS-1, NBS-2, NBS-3, PYRIN-12/NBS-4, NBS-5, PYRIN-1, PYRIN-2, PYRIN-3, PYRIN-5, PYRIN-6, PYRIN-7, PYRIN-8, PYRIN-IO, and PYRIN-11.

An LRR domain usually is composed of several leucine rich repeats, which typically are involved in protein-protein interactions.

Without being bound by a particular theory, it is possible that the LRR domain of PYRIN-9 interacts with an upstream signaling molecule that is associated with stress, infection, and/or inflammation. This interaction triggers a conformational change in PYRIN-9 that exposes an effector domain, e.g., the pyrin domain of PYRIN-9. The exposed effector domain then mediates interaction with a downstream signaling molecule or molecules to transmit a stress-related, apoptotic or inflammatory signal. In this model, the conformational change is dependent upon hydrolysis of a nucleotide triphosphate (ATP or GTP) bound to the NBS domain.

PYRIN-9 molecules are useful as modulating agents in regulating a variety of cellular processes including cell growth and cell death. In one aspect, this invention provides isolated nucleic acid molecules encoding PYRIN-9 proteins or biologically active portions thereof, as well as nucleic acid fragments suitable as primers or hybridization probes for the detection of PYRIN-9 encoding nucleic acids.

PYRIN-9 polypeptides, nucleic acids and modulators of PYRIN-9 expression or activity can be useful for modulating an inflammatory response, modulating NF-κB activation, modulating an apoptotic response, treating inflammatory disorders, e.g., auto-inflammatory disorders and disorders associated with an inappropriate level of apoptosis, and treating immune system disorders. The inflammatory and immune disorders include, but are not limited to, chronic inflammatory diseases and disorders and/or inflammatory bowel disorders, such as Crohn's disease, ulcerative colitis, reactive arthritis, rheumatoid arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, respiratory inflammatory diseases and disorders, such as asthma and chronic obstructive pulmonary disease, gastrointestinal allergies, including food allergies, eosinophilia, conjunctivitis, glomerular nephritis, certain pathogen susceptibilities such as helminthic (e.g., leishmaniasis), certain viral infections, including HIV, and bacterial infections, including tuberculosis and lepromatous leprosy.

Ischemia is often accompanied by inflammation that causes cell death. Because PYRIN-9 molecules are expected to play a role in stress-related response, inflammation and apoptosis, PYRIN-9 polypeptides, nucleic acids, and modulators of PYRIN-9 expression or activity can be used to treat cells death accompanying inflammatory responses triggered by ischemia.

Invasive infection with Gram-negative bacteria and Gram-positive bacteria often results in septic shock. PYRIN-9 may recognize and bind components of Gram-negative bacteria and Gram-positive bacteria or other infectious agents (e.g., intracellular parasites), triggering an inflammatory response. Thus, PYRIN-9 may play a role in innate immune system responses that is similar to that of Toll-like receptor 2 (TLR2), a receptor which has some structural similarity to plant R proteins and IL-1R. TLR2 is a signaling receptor that, in association with CD14, is activated by LPS in a response that requires LPS-binding protein. The interaction of TLR2 with LPS leads to TLR2 oligomerization and recruitment of IRAK (Yang et al. (1998) Nature 395:284-88; Yang et al (1999) J. Immunol. 163:639-43; and Yoshimura et al. (1999) J. Immunol. 163:105). Thus, TLR2 is thought to be a direct mediator of signaling by LPS. TLR2 is also thought to mediate cell activation induced by peptidoglycan and lipoteichoic acid, the main stimulatory components of Gram-positive bacteria (Schwandner et al. (1999) J. Biol. Chem. 274:17406-09).

In addition to the aforementioned disorders, PYRIN-9 polypeptides, nucleic acids, and modulators of PYRIN-9 expression or activity can be used to treat septic shock and other disorders associated with an innate immune response. For example, PYRIN-9 may bind to a component of an intracellular infectious agent or a component of an infectious agent that is brought into a cell expressing PYRIN-9, e.g., a component that enters a cell through a receptor or is expressed by a viral gene.

The invention encompasses methods of diagnosing and treating patients who are suffering from a disorder associated with an abnormal level or rate (undesirably high or undesirably low) of apoptotic cell death, abnormal activity of stress-related pathways of the endoplasmic reticulum (ER), abnormal activity of the Fas/APO-1 receptor complex, abnormal activity of the TNF receptor complex, or abnormal activity of a caspase by administering a compound that modulates the expression of PYRIN-9 (at the DNA, mRNA or protein level, e.g., by altering mRNA splicing) or by altering the activity of PYRIN-9. Examples of such compounds include small molecules, antisense nucleic acid molecules, ribozymes, and polypeptides.

Certain disorders are associated with an increased number of surviving cells, which are produced and continue to survive or proliferate when apoptosis is inhibited or occurs at an undesirably low rate. PYRIN-9 molecules and compounds that modulate the expression or activity of PYRIN-9 can be used to treat or diagnose such disorders. These disorders include cancer (particularly follicular lymphomas, chronic myelogenous leukemia, melanoma, colon cancer, lung carcinoma, carcinomas associated with mutations in p53, and hormone-dependent tumors such as breast cancer, prostate cancer, and ovarian cancer). Such compounds can also be used to treat infections such as infections by bacteria, fungus, parasites, or viruses (such as those caused by herpesviruses, poxviruses, and adenoviruses). Failure to remove autoimmune cells that arise during development or that develop as a result of somatic mutation during an immune response can result in autoimmune disease. Thus, an autoimmune disorder can be caused by an undesirably low level of apoptosis. Accordingly, PYRIN-9 and modulators of PYRIN-9 activity or expression can be used to treat autoimmune disorders (e.g., systemic lupus erythematosis, immune-mediated glomerulonephritis, and arthritis).

Many diseases are associated with an undesirably high rate of apoptosis. PYRIN-9 molecules and modulators of PYRIN-9 expression or activity can be used to treat or diagnose such disorders. A wide variety of neurological diseases are characterized by the gradual loss of specific sets of neurons. Such disorders include Alzheimer's disease, Parkinson's disease, amyotrophic lateral sclerosis (ALS), retinitis pigmentosa, spinal muscular atrophy, Huntington's disease, and various forms of cerebellar degeneration. The cell loss in these diseases does not induce an inflammatory response, and apoptosis appears to be the mechanism of cell death. In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses. Two common disorders associated with cell death are myocardial infarction and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis. Additional diseases associated with an undesirably high rate of apoptosis include: ischemic and hypoxic brain injury, traumatic and excitotoxic brain damage, neuronal transplantation, acute bacterial meningitis, kidney ischemia/reperfusion injury, and liver disease. PYRIN-9 and modulators of PYRIN-9 may therefore be useful in treating and diagnosing these conditions.

Populations of cells are often depleted in the event of viral infection, with perhaps the most dramatic example being the cell depletion caused by the human immunodeficiency virus (HIV). Surprisingly, most T cells that die during HIV infections do not appear to be infected with HIV. Although a number of explanations have been proposed, recent evidence suggests that stimulation of the CD4 receptor results in the enhanced susceptibility of uninfected T cells to undergo apoptosis.

In addition to the aforementioned disorders, PYRIN-9 polypeptides, nucleic acids, and modulators of PYRIN-9 expression or activity can be used to treat disorders of cell signaling and disorders of tissues in which PYRIN-9 is expressed.

The invention features a nucleic acid molecule which is at least 45% (or 55%, 65%, 75%, 85%, 95%, 98% or 99%) identical to the nucleotide sequence shown in SEQ ID NO:1, or a complement thereof.

The invention features a nucleic acid molecule which includes a fragment of at least 150 (300, 325, 350, 375, 400, 425, 450, 500, 550, 600, 650, 700, 800, 900, 1000, 1100, 1200, 1300, 1400, 1500, 1600, 1800, 2000, 2250, 2500, or 2600) nucleotides of the nucleotide sequence shown in SEQ ID NO:1, or a complement thereof.

The invention also features a nucleic acid molecule that hybridizes to a nucleic acid molecule consisting of the nucleotide sequence of SEQ ID NO:1, or a complement thereof, under conditions of incubation at 45° C. in 6.0×SSC followed by washing in 0.2×SSC/0.1% SDS at 65° C.

In an embodiment, a PYRIN-9 nucleic acid molecule has the nucleotide sequence shown in SEQ ID NO:1.

Also within the invention is a nucleic acid molecule which encodes a fragment of a polypeptide having the amino acid sequence of SEQ ID NO:2.

The invention includes a nucleic acid molecule which encodes a naturally occurring allelic variant of a polypeptide comprising the amino acid sequence of SEQ ID NO:2, wherein the nucleic acid molecule hybridizes to a nucleic acid molecule consisting of SEQ ID NO:1 under stringent conditions.

In general, an allelic variant of a gene will be readily identifiable as mapping to the same chromosomal location as the gene.

The invention also includes a nucleic acid molecule encoding a naturally occurring polypeptide, wherein the nucleic acid hybridizes to a nucleic acid molecule consisting of SEQ ID NO:1 under stringent conditions (e.g., hybridization in 6× sodium chloride/sodium citrate (SSC) at about 60° C., followed by one or more washes in 0.2×SSC, 0.1% SDS at 65° C.). Thus, the invention encompasses a nucleic acid molecule which includes the sequence of the protein coding region of a naturally occurring mRNA (or the corresponding cDNA sequence) that is expressed in a human cell.

Also within the invention are: an isolated PYRIN-9 protein having an amino acid sequence that is at least about 65%, preferably 75%, 85%, 95%, 98% or 99% identical to the amino acid sequence of SEQ ID NO:2; an isolated PYRIN-9 protein having an amino acid sequence that is at least about 65%, preferably 75%, 85%, 95%, 98% or 99% identical to the pyrin domain of SEQ ID NO:2 (e.g., about amino acid residues 22-96 of SEQ ID NO:2); an isolated PYRIN-9 protein having an amino acid sequence that is at least about 65%, preferably 75%, 85%, 95%, 98% or 99% identical to the NBS domain of SEQ ID NO:2 (e.g., about amino acids 194-513 of SEQ ID NO:2); an isolated PYRIN-9 protein having an amino acid sequence that is at least about 65%, preferably 75%, 85%, 95%, 98% or 99% identical to the kinase 1a domain of SEQ ID NO:2 (e.g., about amino acids 194-219 of SEQ ID NO:2); an isolated PYRIN-9 protein having an amino acid sequence that is at least about 65%, preferably 75%, 85%, 95%, 98% or 99% identical to the Motif II domain of SEQ ID NO:2 (e.g., about amino acids 226-256 of SEQ ID NO:2); an isolated PYRIN-9 protein having an amino acid sequence that is at least about 65%, preferably 75%, 85%, 95%, 98% or 99% identical to the kinase 2 domain of SEQ ID NO:2 (e.g., about amino acids 260-284 of SEQ ID NO:2); an isolated PYRIN-9 protein having an amino acid sequence that is at least about 65%, preferably 75%, 85%, 95%, 98% or 99% identical to the kinase 3a domain of SEQ ID NO:2 (e.g., about amino acids 304-329 of SEQ ID NO:2); an isolated PYRIN-9 protein having an amino acid sequence that is at least about 65%, preferably 75%, 85%, 95%, 98% or 99% identical to the Motif V domain of SEQ ID NO:2 (e.g., about amino acids 380-400 of SEQ ID NO:2); an isolated PYRIN-9 protein having an amino acid sequence that is at least about 65%, preferably 75%, 85%, 95%, 98% or 99% identical to the Motif VI domain of SEQ ID NO:2 (e.g., about amino acids 467-483 of SEQ ID NO:2); an isolated PYRIN-9 protein having an amino acid sequence that is at least about 65%, preferably 75%, 85%, 95%, 98% or 99% identical to the Motif VII domain of SEQ ID NO:2 (e.g., about amino acids 494-513 of SEQ ID NO:2); an isolated PYRIN-9 protein having an amino acid sequence that is at least about 65%, preferably 75%, 85%, 95%, 98% or 99% identical to the LRR domain of SEQ ID NO:2 (e.g., about amino acids 729-892 of SEQ ID NO:2); and an isolated PYRIN-9 protein having an amino acid sequence that is at least about 65%, preferably 75%, 85%, 95%, 98% or 99% identical to one or more of the leucine rich repeats of SEQ ID NO:2 (e.g., about amino acids 729-754, 755-777, 811-838, or 840-863 of SEQ ID NO:2).

Also within the invention are: an isolated PYRIN-9 protein which is encoded by a nucleic acid molecule having a nucleotide sequence that is at least about 65%, preferably 75%, 85%, 95%, 98% or 99% identical to SEQ ID NO:1; an isolated PYRIN-9 protein which is encoded by a nucleic acid molecule having a nucleotide sequence at least about 65% preferably 75%, 85%, 95%, 98% or 99% identical to the pyrin domain encoding portion of SEQ ID NO:1 (e.g. about nucleotides 64-288 of SEQ ID NO:1); an isolated PYRIN-9 protein which is encoded by a nucleic acid molecule having a nucleotide sequence at least about 65% preferably 75%, 85%, 95%, 98% or 99% identical to the NBS domain encoding portion of SEQ ID NO:1 (e.g. about nucleotides 580-1539 of SEQ ID NO:1); an isolated PYRIN-9 protein which is encoded by a nucleic acid molecule having a nucleotide sequence at least about 65% preferably 75%, 85%, 95%, 98% or 99% identical to the kinase 1a, Motif II, kinase 2, kinase 3a region, Motif V, Motif VI, or Motif VII encoding portion of SEQ ID NO:1 (e.g. about nucleotides 580-657, 676-768, 778-852, 910-987, 1138-1200, 1399-1449, 1480-1539 of SEQ ID NO:1, respectively); an isolated PYRIN-9 protein which is encoded by a nucleic acid molecule having a nucleotide sequence at least about 65% preferably 75%, 85%, 95%, 98% or 99% identical to the LRR domain encoding portion of SEQ ID NO:1 (e.g. about nucleotides 2185-2676 of SEQ ID NO:1) or one or more leucine rich repeat encoding portions of SEQ ID NO:1 (e.g. about nucleotides 2185-2262, 2263-2331, 2431-2514 or 2518-2589 of SEQ ID NO:1); and an isolated PYRIN-9 protein which is encoded by a nucleic acid molecule having a nucleotide sequence which hybridizes under stringent hybridization conditions to a nucleic acid molecule having the nucleotide sequence of SEQ ID NO:1.

The PYRIN-9 nucleic acids, polypeptides, and antibodies of the invention may be useful for mapping the location of the PYRIN-9 gene.

Another embodiment of the invention features PYRIN-9 nucleic acid molecules which specifically detect PYRIN-9 nucleic acid molecules, relative to nucleic acid molecules encoding other members of the PYRIN/NBS/LRR superfamily. For example, in one embodiment, a PYRIN-9 nucleic acid molecule hybridizes under stringent conditions to a nucleic acid molecule comprising the nucleotide sequence of SEQ ID NO:1, or a complement thereof. In another embodiment, the PYRIN-9 nucleic acid molecule is at least 300 (350, 400, 450, 500, 550, 600, 650, 700, 800, 900, 1000, 1100, 1200, 1300, 1400, 1500, 1600, 1800, 2000, 2250, 2500, or 2600) nucleotides in length and hybridizes under stringent conditions to a nucleic acid molecule comprising the nucleotide sequence shown in SEQ ID NO:1, or a complement thereof. In another embodiment, an isolated PYRIN-9 nucleic acid molecule comprises the pyrin domain encoding portion of SEQ ID NO:1 or a complement thereof. In another embodiment, an isolated PYRIN-9 nucleic acid molecule comprises the NBS domain encoding portion of SEQ ID NO:1 or a complement thereof. In another embodiment, an isolated PYRIN-9 nucleic acid molecule comprises the LRR domain encoding portion of SEQ ID NO:1 or a complement thereof. In yet another embodiment, the invention provides an isolated nucleic acid molecule which is antisense to the coding strand of a PYRIN-9 nucleic acid.

Another aspect of the invention provides a vector, e.g., a recombinant expression vector, comprising a PYRIN-9 nucleic acid molecule of the invention. In another embodiment the invention provides a host cell containing such a vector. The invention also provides a method for producing PYRIN-9 protein by culturing, in a suitable medium, a host cell of the invention containing a recombinant expression vector such that a PYRIN-9 protein is produced.

Another aspect of this invention features isolated or recombinant PYRIN-9 proteins and polypeptides. Preferred PYRIN-9 proteins and polypeptides possess at least one biological activity possessed by naturally occurring human PYRIN-9, e.g., (1) the ability to form protein:protein interactions with proteins in an apoptotic and/or inflammatory signaling pathway; (2) the ability to form pyrin domain-pyrin domain interactions with proteins in an apoptotic and/or inflammatory signaling pathway; (3) the ability to bind to and/or hydrolyze a nucleotide, e.g., ATP or GTP; (4) the ability to bind a PYRIN-9 ligand; and (5) the ability to bind to an intracellular target. Other activities include: (1) modulation of cellular proliferation; (2) modulation of cellular differentiation; (3) modulation of cellular death; (4) modulation of ER-specific apoptosis pathways; (5) modulation of amyloid-α-mediated neurotoxicity; (6) modulation of the NF-κB pathway; and (7) modulation of stress-responsive signaling pathways.

The PYRIN-9 proteins of the present invention, or biologically active portions thereof, can be operatively linked to a non-PYRIN-9 polypeptide (e.g., heterologous amino acid sequences) to form PYRIN-9 fusion proteins, respectively. The invention further features antibodies that specifically bind PYRIN-9 proteins, such as monoclonal or polyclonal antibodies. In addition, the PYRIN-9 proteins or biologically active portions thereof can be incorporated into pharmaceutical compositions, which optionally include pharmaceutically acceptable carriers.

In another aspect, the present invention provides a method for detecting the presence of PYRIN-9 activity or expression in a biological sample by contacting the biological sample with an agent capable of detecting an indicator of PYRIN-9 activity such that the presence of PYRIN-9 activity is detected in the biological sample.

In another aspect, the invention provides a method for modulating PYRIN-9 activity comprising contacting a cell with an agent that modulates (inhibits or stimulates) PYRIN-9 activity or expression such that PYRIN-9 activity or expression in the cell is modulated. In one embodiment, the agent is an antibody that specifically binds to PYRIN-9 protein. In another embodiment, the agent modulates expression of PYRIN-9 by modulating transcription of a PYRIN-9 gene, splicing of a PYRIN-9 mRNA, or translation of a PYRIN-9 mRNA. In yet another embodiment, the agent is a nucleic acid molecule having a nucleotide sequence that is antisense to the coding strand of the PYRIN-9 mRNA or the PYRIN-9 gene.

In one embodiment, the methods of the present invention are used to treat a subject having a disorder characterized by aberrant PYRIN-9 protein or nucleic acid expression or activity or related to PYRIN-9 expression or activity by administering an agent which is a PYRIN-9 modulator to the subject. In one embodiment, the PYRIN-9 modulator is a PYRIN-9 protein. In another embodiment the PYRIN-9 modulator is a PYRIN-9 nucleic acid molecule. In other embodiments, the PYRIN-9 modulator is a peptide, peptidomimetic, or other small molecule.

The present invention also provides a diagnostic assay for identifying the presence or absence of a genetic lesion or mutation characterized by at least one of: (i) aberrant modification or mutation of a gene encoding a PYRIN-9 protein; (ii) mis-regulation of a gene encoding a PYRIN-9 protein; (iii) aberrant RNA splicing; and (iv) aberrant post-translational modification of a PYRIN-9 protein, wherein a wild-type form of the gene encodes a protein with a PYRIN-9 activity.

In another aspect, the invention provides a method for identifying a compound that binds to or modulates the activity of a PYRIN-9 protein. In general, such methods entail measuring a biological activity of a PYRIN-9 protein in the presence and absence of a test compound and identifying those compounds that alter the activity of the PYRIN-9 protein.

The invention also features methods for identifying a compound that modulates the expression of PYRIN-9 by measuring the expression of PYRIN-9 in the presence and absence of a compound.

The invention also features methods for identifying a compound that alters (increases or decreases) the binding of PYRIN-9 (or a pyrin domain-, NBS domain-, or LRR domain containing portion thereof) to another protein (e.g., a PYRIN-9 protein or other pyrin domain-containing protein, e.g. CARD-5) or molecule. For example, the method includes measuring the binding of the protein (or polypeptide or molecule) to each other in the presence and absence of a test compound and identifying the test compound as a compound that alters binding if the binding in the presence of test compound differs from the binding in the absence of the test compound.

The invention also features a method for identifying a compound that binds to the NBS domain of PYRIN-9 by measuring the binding of a test compound to a polypeptide comprising the NBS domain of PYRIN-9. The binding can be measured in the presence of a nucleotide (e.g., an NTP such as ATP or GTP) for a competitive binding assay. Alternatively, the binding can be measured in the absence of a nucleotide that binds to the NBS site.

The invention also features a method for identifying a candidate compound for modulating (increasing or decreasing) the binding of PYRIN-9 to CARD-5, the method including: (a) measuring the binding of a first polypeptide comprising the pyrin domain of CARD-5 to a second polypeptide comprising the pyrin domain of PYRIN-9 in the presence of a test compound; and (b) comparing the binding of the first polypeptide to the second polypeptide measured in step (a) to the binding of the first polypeptide to the second polypeptide in the absence of the test compound, wherein altered binding of the first polypeptide to the second polypeptide in the presence of the test compound compared the binding in the absence of the test compound indicates that the test compound modulates the binding of PYRIN-9 to CARD-5. The binding of PYRIN-9 to CARD-5 can be detected directly or indirectly (e.g., by detecting a biological response that results from the interaction of PYRIN-9 and CARD-5, such as NF-κB activation and/or caspase-1 activation).

In another embodiment the method includes a method for identifying a candidate compound for modulating the CARD-5-mediated activation of NF-κB, the method comprising: a) measuring the binding of a first polypeptide comprising the pyrin domain of CARD-5 to a second polypeptide comprising the pyrin domain of PYRIN-9 in the presence of a test compound; and b) comparing the binding of the first polypeptide to the second polypeptide measured in step (a) to the binding of the first polypeptide to the second polypeptide in the absence of the test compound, wherein altered binding of the first polypeptide to the second polypeptide in the presence of the test compound compared the binding in the absence of the test compound indicates that the test compound is candidate compound for modulating the CARD-5-mediated activation of NF-κB.

In another embodiment the invention features a method for identifying a modulator of NF-κB activity, the method comprising: a) providing a cell expressing recombinant CARD-5 and recombinant PYRIN-9; b) exposing the cell to a test compound; and c) measuring the NF-κB activity of the cell in the presence of the test compound, wherein altered activation in the presence of the test compound compared to the absence of the test compound indicates that the compound is a modulator of NF-κB activity.

In another embodiment the invention features a method for identifying a candidate compound for modulating the CARD-5-mediated activation of NF-κB, the method comprising: a) measuring the binding of a test compound to the LRR domain of PYRIN-9; and b) determining that the test compound is a candidate compound for modulating the CARD-5-mediated activation of NF-κB when the test compound binds to the LRR domain of PYRIN-9.

In another embodiment the invention features a method for identifying a candidate modulator of PYRIN-9, the method comprising: a) contacting a purified polypeptide comprising the NBS domain of PYRIN-9 with a test compound in the presence of a nucleotide that binds to the NBS domain in the absence of the test compound; b) measuring the binding of the nucleotide to the NBS domain in the presence of the test compound; and c) identifying the test compound as a candidate modulator of PYRIN-9 if the test compound reduces the binding of the nucleotide to the NBS domain. In various embodiments: the nucleotide is bound to the NBS domain before the polypeptide is exposed to the test compound, the test compound is exposed to the polypeptide before the polypeptide is exposed to the nucleotide, the nucleotide is selected from the group consisting of an adenine nucleotide, a guanidine nucleotide, a thymidine nucleotide, a cytosine nucleotide, and a uridine nucleotide, the nucleotide is selected from the group consisting of a ribonucleotide and a dideoxribonucleotide, and the nucleotide is selected from the group including, but not limited to ATP, ADP, TTP, TDP, UTP, UDP, CTP, CDP, GTP, and GDP.

In another embodiment the invention features a method for identifying a candidate modulator of PYRIN-9, the method comprising: a) contacting a purified polypeptide comprising the NBS domain of PYRIN-9 with a test compound in the presence of a nucleotide triphosphate that binds to the NBS domain in the absence of the test compound; b) measuring the hydrolysis of the nucleotide triphosphate in the presence of the test compound; and c) identifying the test compound is a candidate modulator of PYRIN-9 if the test compound modulates (e.g. reduces or increases) the hydrolysis of the nucleotide triphosphate.

The invention also features a method for identifying an inhibitor of caspase activity, the method including: (a) providing a cell expressing recombinant CARD-5 and recombinant PYRIN-9; (b) exposing the cell to a test compound; and (c) measuring the caspase activity, e.g., caspase-1 activity, of the cell in the presence of the test compound.

The invention also features a method for identifying a compound that modulates the ability of PYRIN-9 to activate caspase-1, the method including: (a) contacting a polypeptide comprising SEQ ID NO:2 or a fragment thereof (e.g., a fragment including a domain described herein) with a test compound; and (b) determining the effect of the test compound on the ability of the polypeptide to activate caspase-1.

The invention also features methods for treating disorders associated with inappropriate apoptosis (e.g., Alzheimer's diseases or other neurological disorders associated with neuronal apoptosis, cancer or inflammation) by modulating the expression or activity of PYRIN-9.

In one example, the invention features a method of treating a disorder associated with inappropriate apoptosis, including the steps of: selecting an individual that has a disorder associated with inappropriate apoptosis; and modulating the expression or activity of a polypeptide containing the amino acid sequence of SEQ ID NO:2.

In another example, the invention features a method of treating cancer, including the steps of: selecting an individual that has cancer; and modulating the expression or activity of a polypeptide containing the amino acid sequence of SEQ ID NO:2.

In another example, the invention features a method of treating an inflammatory disorder, including the steps of: selecting an individual that has an inflammatory disorder; and modulating the expression or activity of a polypeptide containing the amino acid sequence of SEQ ID NO:2.

The invention also features a method for identifying a candidate compound for modulating the binding of PYRIN-9 to CARD-5, the method comprising: a) measuring the binding of a first polypeptide comprising the pyrin domain of CARD-5 to a second polypeptide comprising the pyrin domain of PYRIN-9 in the presence of a test compound; and b) comparing the binding of the first polypeptide to the second polypeptide measured in step (a) to the binding of the first polypeptide to the second polypeptide in the absence of the test compound, wherein altered binding of the first polypeptide to the second polypeptide in the presence of the test compound compared the binding in the absence of the test compound indicates that the test compound modulates the binding of PYRIN-9 to CARD-5.

In another embodiment the method includes a method for identifying a candidate compound for modulating the CARD-5-mediated activation of NF-κB, the method comprising: a) measuring the binding of a first polypeptide comprising the pyrin domain of CARD-5 to a second polypeptide comprising the pyrin domain of PYRIN-9 in the presence of a test compound; and b) comparing the binding of the first polypeptide to the second polypeptide measured in step (a) to the binding of the first polypeptide to the second polypeptide in the absence of the test compound, wherein altered binding of the first polypeptide to the second polypeptide in the presence of the test compound compared the binding in the absence of the test compound indicates that the test compound is candidate compound for modulating the CARD-5-mediated activation of NF-κB.

In another embodiment the invention features a method for identifying an modulator of NF-κB activity, the method comprising: a) providing a cell expressing recombinant CARD-5 and recombinant PYRIN-9; b) exposing the cell to a test compound; and c) measuring the NF-κB activity of the cell in the presence of the test compound, wherein altered activation in the presence of the test compound compared to the absence of the test compound indicates that the compound is a modulator of NF-κB activity.

In another embodiment the invention features a method for identifying a candidate compound for modulating the CARD-5-mediated activation of NF-κB, the method comprising: a) measuring the binding of a test compound to the LRR domain of PYRIN-9; and b) determining that the test compound is a candidate compound for modulating the CARD-5-mediated activation of NF-κB when the test compound binds to the LRR domain of PYRIN-9.

In another embodiment the invention features a method for identifying a candidate compound for modulating the CARD-5-mediated activation of NF-κB, the method comprising: a) measuring the binding of a test compound to the LRR domain of PYRIN-9; and b) identifying the test compound as an LRR domain binding compound when the test compound binds to the LRR domain of PYRIN-9; c) measuring the activation of NF-κB in a cell expressing CARD-5 and PYRIN-9 in the presence and absence of the LRR domain binding compound, wherein the LRR domain binding compound is a candidate compound for modulating the CARD-5-mediated activation of NF-κB if the activation of NF-κB in the presence of the LRR domain binding compound is greater than in the absence of the LRR domain binding compound.

In another embodiment the invention features a method for identifying a candidate modulator of PYRIN-9, the method comprising: a) contacting a purified polypeptide comprising the NBS domain of PYRIN-9 with a test compound in the presence of a nucleotide that binds to the NBS domain in the absence of the test compound; b) measuring the binding of the nucleotide to the NBS domain in the presence of the test compound; and c) identifying the test compound as a candidate modulator of PYRIN-9 if the test compound reduces the binding of the nucleotide to the NBS domain. In various embodiments: the nucleotide is bound to the NBS domain before the polypeptide is exposed to the test compound, the test compound is exposed to the polypeptide before the polypeptide is exposed to the nucleotide, the nucleotide is selected from the group consisting of an adenine nucleotide, a guanidine nucleotide, a thymidine nucleotide, a cytosine nucleotide, and a uridine nucleotide, the nucleotide is selected from the group consisting of a ribonucleotide and a dideoxribonucleotide, and the nucleotide is selected from the group including, but not limited to ATP, ADP, TTP, TDP, UTP, UDP, CTP, CDP, GTP, and GDP.

In another embodiment the invention features a method for identifying a candidate modulator of PYRIN-9, the method comprising: a) contacting a purified polypeptide comprising the NBS domain of PYRIN-9 with a test compound in the presence of a nucleotide that binds to the NBS domain in the absence of the test compound; b) measuring the binding of the nucleotide to the NBS domain in the presence of the test compound; c) identifying a test compound that reduces the binding of the nucleotide to the NBS domain; and d) measuring the binding of a test compound that reduces the binding of the nucleotide to the NBS domain of PYRIN-9 to an NBS domain of a protein other than PYRIN-9; wherein the test compound is a candidate modulator of PYRIN-9 if it reduces the binding of the nucleotide to the NBS domain of PYRIN-9 and does not substantially reduce the binding of the nucleotide of the NBS domain of a protein other than PYRIN-9. In certain embodiments, the NBS of a protein other than PYRIN-9 is the NBS domain of a protein selected from the group consisting of: CARD-12, CARD-4, NBS-1, NBS-2, NBS-3, PYRIN-12/NBS-4, NBS-5, PYRIN-1, PYRIN-2, PYRIN-3, PYRIN-5, PYRIN-6, PYRIN-7, PYRIN-8, PYRIN-10, and PYRIN-11.

In another embodiment the invention features a method for identifying a candidate modulator of PYRIN-9, the method comprising: a) contacting a purified polypeptide comprising the NBS domain of PYRIN-9 with a test compound in the presence of a nucleotide triphosphate that binds to the NBS domain in the absence of the test compound; b) measuring the hydrolysis of the nucleotide triphosphate in the presence of the test compound; and c) identifying the test compound is a candidate modulator of PYRIN-9 if the test compound reduces the hydrolysis of the nucleotide triphosphate.

In another embodiment the invention features a method for identifying a modulator of caspase-1 activity, the method including: a) providing a cell expressing recombinant PYRIN-9; b) exposing the cell to a test compound; and c) measuring caspase-1 activity of the cell in the presence of the test compound, wherein altered caspase-1 activity in the presence of the test compound compared to the absence of the test compound indicates that the compound is a modulator of caspase-1 activity.

A fragment of PYRIN-9, e.g., a fragment containing the pyrin domain, the NBS domain or the LRR domain can be used in the methods described herein. In addition to recombinant PYRIN-9 or a fragment thereof, a cell can also express recombinant CARD-5, caspase-1, and/or a fragment of CARD-5 or caspase-1. Caspase-1 activity can be detected, for example, by measuring the presence of protein products produced by caspase-1 proteolytic activity (e.g., detecting IL-1β secretion).

The method can also include an additional step of measuring the caspase-1 activity of the cell in the absence of the test compound and/or in the absence of PYRIN-9 (e.g., recombinant PYRIN-9).

In another embodiment the invention features a method for identifying a candidate modulator of PYRIN-9, the method including: a) contacting a purified polypeptide containing the pyrin domain of PYRIN-9 with a test compound in the presence of caspase-1 and a caspase-1 substrate; b) measuring the proteolysis of the caspase-1 substrate in the presence of the test compound; and c) identifying the test compound as a candidate modulator of PYRIN-9 if the test compound modulates the proteolysis of the caspase-1 substrate. For example, the caspase-1 substrate can be an unprocessed form of a cytokine, e.g., pro-IL-1β, wherein caspase-1 proteolysis results in the formation of a bioactive form of the cytokine, e.g., bioactive IL-1β.

The method can also include an additional step of measuring the proteolysis of the caspase-1 substrate in the absence of the test compound and/or comparing the proteolysis of the caspase-1 substrate in the absence of the test compound with that measured in the presence of the test compound.

The invention also features a method for modulating CARD-5 activity in a patient, the method comprising administering a compound that alters the activity of PYRIN-9, a method for modulating NF-κB activity in a patient, the method comprising administering a compound that alters the activity of PYRIN-9, and a method for treating an inflammatory disorder in a patient, the method comprising administering a compound that alters the activity of PYRIN-9.

The above described methods can also be used for identifying a candidate compound for treating an inflammatory disorder.

Other features and advantages of the invention will be apparent from the following detailed description and claims.

### BRIEF DESCRIPTION OF THE DRAWINGS

FIGS. 1A-1D are graphs depicting: (A) PYRIN-9 (PYPAF5) synergizing with CARD-5 (ASC) to induce NF-κB activation; (B) the concentration dependent activation of NF-κB by PYRIN-9 (PYPAF5); (C) the importance of the N-terminal PYRIN domain of PYRIN-9 (PYPAF5) in synergizing with CARD-5 (ASC) to induce NF-κB activation; and (D) the activation of NF-κB activity by PYRIN-9 (PYPAF5) and CARD-5 (ASC) through the IKK complex.

FIGS. 2A-2C are graphs depicting: (A) the activation of pro-caspase-1 by PYRIN-9 (PYPAF5) and CARD-5 (ASC); (B) the requirement for the N-terminal PYRIN domain of PYRIN-9 (PYPAF5) for the activation of pro-caspase-1; and (C) the dependence of PYRIN-9 (PYPAF5)/CARD-5 (ASC) induction of IL-1β secretion on active caspase-1.

### DETAILED DESCRIPTION OF THE INVENTION

Human PYRIN-9

The present invention is based, in part, on the identification of a sequence encoding a human PYRIN-9 protein. A predicted PYRIN-9 cDNA sequence is represented in SEQ ID NO:1 and a predicted 892 amino acid PYRIN-9 protein represented in SEQ ID NO:2.

PYRIN-9 has a tripartite structure consisting of an N-terminal PYRIN domain, a central NBS domain, and a C-terminal domain comprised of at least four leucine rich repeat (LRR) motifs. The NBS domain belongs to the NACHT subfamily of NTPases and contains seven signature motifs, including the P-loop. The PYRIN domain of PYRIN-9 shares significant sequence similarity with the PYRIN domains of other PYRIN-containing proteins.

A detailed analysis of the predicted PYRIN-9 amino acid sequence showed it to contain a pyrin domain (e.g., about amino acid residues 22-96 of SEQ ID NO:2), a nucleotide binding site (NBS; e.g., about amino acid residues 194-513 of SEQ ID NO:2), and several leucine rich repeats which form a LRR domain (e.g., about amino acid residues 729-892 of SEQ ID NO:2). Within the predicted NBS there is a kinase 1a domain (Motif I; P-loop) (e.g., about amino acid residues 194-219 of SEQ ID NO:2), a Motif II domain (e.g., about amino acid residues 226-256 of SEQ ID NO:2), a kinase 2 domain (Motif III; Walker B box) (e.g., about amino acid residues 260-284 of SEQ ID NO:2), a kinase 3a domain (Motif IV) (e.g., about amino acid residues 304-329 of SEQ ID NO:2), a Motif V domain (e.g., about amino acid residues 380-400 of SEQ ID NO:2), a Motif VI domain (e.g., about amino acid residues 467-483 of SEQ ID NO:2), and a Motif VII domain (e.g., about amino acid residues 494-513 of SEQ ID NO:2).

Further analysis of the predicted PYRIN-9 amino acid sequence showed it to contain several potential sites of phosphorylation. For example, phosphorylation sites of PYRIN-9 include, but are not limited to, at least one, preferably two cAMP- or cGMP-dependent protein kinase phosphorylation sites at about amino acid residues 145-148 (phosphorylation on threonine) and 686-689 (phosphorylation on serine) of SEQ ID NO:2; at least one, two, three, four, five, six, seven preferably eight casein kinase II phosphorylation sites at about amino acid residues 628-631, 815-818, (phosphorylation on threonine), 114-117, 248-251, 350-353, 481-484, 501-504 and 857-860 (phosphorylation on serine) of SEQ ID NO:2; at least one, two, three, four, five, six, seven, eight, nine, ten, eleven, preferably twelve protein kinase C phosphorylation sites at about amino acid residues 84-86, 144-146, 325-327, 411-413, 543-545, 707-709, 789-791 (phosphorylation on threonine) 140-142, 350-352, 481-483, 568-570, and 676-678 (phosphorylation on serine) of SEQ ID NO:2; or at least one tyrosine phosphorylation site at about amino acid residues 366-372 of SEQ ID NO:2. Signal transduction processes frequently involve modulating the activity, e.g. activation or deactivation, of at least one molecule in a signal transduction pathway by the action of a least one enzyme, e.g. a kinase and/or a phosphatase, to modify the phosphorylation state, e.g. phosphorylate or dephosphorylate, the molecule in the signal transduction pathway. Accordingly, the activity of PYRIN-9 can be modulated by at least one molecule capable of phosphorylating or dephosphorylating a PYRIN-9 polypeptide.

A role for PYRIN-9 in inflammatory signaling is indicated by the finding that mutations in the gene encoding the related PYRIN-1 (PYPAF1) protein are associated with Muckle-Wells syndrome and familial cold urticaria, two genetically determined auto-inflammatory disorders that are similar to the rare inflammatory disorder familial Mediterranean fever (Hoffman et al. (2002) Nat. Genet. 29, 301-305). PYRIN-9 also shares homology with pyrin, a protein that causes familial Mediterranean fever (Bertin et al. (2000) Cell Death Differ. 7:1273-74).

As detailed herein, PYRIN-9 expression was found to be largely restricted to immune cells. Co-expression of PYRIN-9 with CARD-5 resulted in the recruitment of PYRIN-9 to distinct cytoplasmic loci and induced a potent synergistic activation of NF-κB activity. In addition, co-expression of PYRIN-9 and CARD-5 resulted in the synergistic activation of caspase-1 and the secretion of interleukin-1β (IL-1β).

The experimental findings presented herein indicate that PYRIN-9 participates in inflammatory signal transduction pathways by regulating caspase-1-dependent cytokine processing and the activation of NF-κB in response to pro-inflammatory stimuli. These findings are consistent with roles that have been identified for other related pyrin-domain containing proteins in inflammatory signaling.

TABLE 1
Summary of Domains of PYRIN-9

| Domain | Location |
|---|---|
| Pyrin domain | about amino acid residues 22-96 of SEQ ID NO:2 |
| NBS domain | about amino acid residues 194-513 of SEQ ID NO:2 |
| Kinase 1a domain (Motif I; | about amino acid residues 194-219 of SEQ ID NO:2 |
| P-loop) | |
| Motif II | about amino acid residues 226-256 of SEQ ID NO:2 |
| Kinase 2 domain | about amino acid residues 260-284 of SEQ ID NO:2 |
| (Motif III; Walker B box) | |
| Kinase 3a domain (Motif IV) | about amino acid residues 304-329 of SEQ ID NO:2 |
| Motif V | about amino acid residues 380-400 of SEQ ID NO:2 |
| Motif VI | about amino acid residues 467-483 of SEQ ID NO:2 |
| Motif VII | about amino acid residues 494-513 of SEQ ID NO:2 |
| LRR domain | about amino acid residues 729-892 of SEQ ID NO:2 |
| leucine rich repeat a | about amino acid residues 729-754 of SEQ ID NO:2 |
| leucine rich repeat b | about amino acid residues 755-777 of SEQ ID NO:2 |
| leucine rich repeat c | about amino acid residues 811-838 of SEQ ID NO:2 |
| leucine rich repeat d | about amino acid residues 840-863 of SEQ ID NO:2 |

Co-Localization of PYRIN-9 and CARD-5

Cellular co-localization studies were carried out to determine whether PYRIN-9 (also known as PYPAF5) interacts with CARD-5 (also known as ASC) when the two proteins were overexpressed in 293T embryonic kidney cells.

Plasmids expressing either full-length PYRIN-9 (PYRIN-9-FL) or a PYRIN-9 truncation mutant lacking the pyrin domain (PYRIN-9-PYRIN) with a C-terminal FLAG epitope were constructed using the pCI mammalian expression vector (Promega, Madison, Wis.).

293T cells were transfected in poly-D-lysine-coated glass chamber slides (BIOCOAT, BD Biosciences, San Jose, Calif.) with plasmids expressing HA-tagged CARD-5 and FLAG-tagged PYRIN-9 using SUPERFECT (transfection reagent, Qiagen, Inc., Valencia, Calif.). Cells were fixed 24 hours after transfection in 4% paraformaldehyde, permeabilized and blocked in a buffer containing 0.3% Triton X-100 (t-Octylphenoxypolyethoxyethanol nonionic detergent, Union Carbide subsidiary of Dow Chemical Co., Midland Mich.), and incubated with the following primary and secondary antibodies: rabbit anti-HA polyclonal Y-11 (Santa Cruz Biotechnology, Inc., Santa Cruz, Calif.), mouse anti-FLAG monoclonal antibody M2 (Sigma-Aldrich, St. Louis, Mo.), Alexa-350 goat anti-rabbit IgG (Molecular Probes, Inc., Eugene, Oreg.), and Alexa-598 goat anti-mouse IgG (Molecular Probes, Inc., Eugene, Oreg.). Images were generated as described previously (Bertin et al. (2001) J. Biol. Chem. 276:11877-82).

FLAG-tagged PYRIN-9 was co-expressed with HA-tagged CARD-5 in cells and detected using a mixture of anti-HA and anti-FLAG antibodies. When expressed alone, PYRIN-9 showed a broad cytoplasmic distribution that excluded the nucleus. As observed previously, CARD-5 localized to cytoplasmic punctate structures when expressed alone (Masumoto et al.(1999) J. Biol. Chem. 274:33835-38). PYRIN-9-FL was found to co-localize with CARD-5 when co-expressed.

The ability of a PYRIN-9 mutant lacking the N-terminal PYRIN domain (PYRIN-9APYRIN) to be recruited to the CARD-5 punctate structures was also examined. When expressed alone, PYRIN-9APYRIN showed a broad cytoplasmic distribution similar to that of PYRIN-9-FL. However, PYRIN-9APYRIN failed to associate with the CARD-5 punctate structures, demonstrating that the PYRIN domain of PYRIN-9 is necessary for recruitment. An interaction between PYRIN-9 and CARD-5 was not detected by performing co-immunoprecipitation experiments when both proteins are overexpressed in cells. This inability to detect an interaction may be due to the relative insolubility of PYRIN-9/CARD-5 complexes that form in the cell when these proteins are co-expressed. Alternatively, the interaction between PYRIN-9 and CARD-5 may be transient thereby precluding its detection by immunoprecipitation analysis. Nonetheless, the co-localization of PYPAF5 with CARD-5 to cytoplasmic loci is required for the synergistic activation of NF-κB and caspase-1-dependent cytokine processing described herein.

CARD-5 is described in U.S. patent application Ser. No. 09/841,879, filed Apr. 24, 2001, U.S. patent application Ser. No. 09/728,721, filed Dec. 1, 2000, and U.S. patent application Ser. No. 09/340,620, filed Jun. 28, 1999, the entire contents of which are incorporated by reference. In addition, CARD-5 sequences are included herein as SEQ ID NOs:3, 4, and 5. SEQ ID NO:3 is the CARD-5 nucleotide coding sequence which encodes the CARD-5 amino acid sequence of SEQ ID NO:4. The pyrin domain of CARD-5 is provided herein as SEQ ID NO:5.

Expression of PYRIN-9

PYRIN-9 gene expression was determined using the Perkin-Elmer/ABI 7700 Sequence Detection System which employs TAQMAN® real-time PCR (Applied Biosystems division of PE Biosystems, Foster City, Calif.) technology. TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of Taq polymerase digests the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a quantitative measure of the initial template concentration. Samples can be internally controlled by the addition of a second set of primers/probe specific for a housekeeping gene such as GAPDH which has been labeled with a different fluorophore on the 5′ end (typically VIC).

To determine the level of PYRIN-9 in various human tissues a primer/probe set was designed. Total RNA was prepared from a series of human tissues using. First strand cDNA was prepared from 1 μg total RNA using an oligo-dT primer and SUPERSCRIPT II reverse transcriptase (Invitrogen Life Technologies, Carlsbad, Calif.). cDNA obtained from approximately 50 ng total RNA was used per TaqMan reaction. Tissues tested include the human tissues and several cell lines shown in Tables 2-4.

As depicted in Tables 2-4, PYRIN-9 mRNA was detected in cell types involved in inflammation and/or immunity. For example, Tables 2-4 show PYRIN-9 to be expressed in granulocytes, T cells, and eosinophils. In CD4, CD8, Th1 and Th2 cells, expression of PYRIN-9 is transiently upregulated with cell activation. The expression of PYRIN-9 in rheumatoid arthritis synovial tissues (RA) is elevated above the levels in normal or osteoarthritis synovium (OA). PYRIN-9 expression also is elevated in chronic obstructive pulmonary disease (COPD) lung tissue compared to normal lung tissue. The relatively restricted expression of PYRIN-9 in immune cells further indicates a role for the protein and its binding-partners in inflammatory signaling.

TABLE 2
Expression of PYRIN-9

| Cell Type | Relative Expression |
|---|---|
| CD4- resting | 2.36 |
| CD4-aCD3/CD28 4/24 h | 2.53 |
| CD8- resting | 1.66 |
| CD8-aCD3/CD28 4/24 h | 3.67 |
| CD14- resting | 0.15 |
| CD14-LPS 4/24 h | 0.14 |
| CD19- resting | 0.22 |
| CD19-LPS 4/24 h | 0.71 |
| CD19-CD40L 4/24 h | 0.35 |
| Macrophage- resting | 0.02 |
| Macrophage-LPS 4/24 h | 0.12 |
| Macrophage-IFNg 4/24 h | 0.09 |
| Macrophage-CD40L 4/24 h | 0.00 |
| Th1-1/6/24 h (46) | 10.25 |
| Th2-1/6/24 h (46) | 5.89 |
| FLS- resting | 0.01 |
| FLS-TNF 4/24 h | 0.00 |
| FLS-IL1 4/24 h | 0.01 |
| NHBE- resting | 0.04 |
| NHBE-IL-4 4/24 h | 0.04 |
| NHBE-IL-13 4/24 h | 0.35 |
| BSMC- resting | 0.43 |
| BSMC-TNF 4/24 h | 0.01 |
| BSMC-IFNg 4/24 h | 0.10 |
| HMVEC- resting | 0.01 |
| HMVEC-IFNg 4/24 h | 0.00 |
| HMVEC-TNF 4/24 h | 0.07 |
| NSYN7/26/01 | 0.33 |
| RASYN7/16/01 | 3.22 |
| OA 7/16/01 | 0.05 |
| Normal Colon pool | 3.55 |
| Colitis Colon pool | 1.77 |
| Crohns Colon pool | 1.36 |
| Normal Brain (pool) | 0.18 |
| Normal Heart | 0.10 |
| Normal Liver | 3.05 |
| Normal Kidney | 24.72 |
| Normal Spleen | 3.99 |
| Normal Tonsil | 3.03 |
| Normal Lymph Node | 7.30 |
| Normal Lung pool | 1.12 |
| COPD-1 | 6.62 |
| COPD-2 | 2.39 |

TABLE 3
Expression of PYRIN-9

| Cell Type | Relative Expression |
|---|---|
| CD4 resting | 0.42 |
| CD4 aCD3 4 hr | 1.38 |
| CD4 aCD3 24 hr | 0.29 |
| CD4 aCD3/CD28 4 hr | 1.86 |
| CD4 aCD3/CD28 24 hr | 0.27 |
| CD8 resting | 0.85 |
| CD8 aCD3 4 hr | 0.79 |
| CD8 aCD3 24 hr | 1.01 |
| CD8 aCD3/CD28 4 hr | 1.41 |
| CD8 aCD3/CD28 24 hr | 0.58 |
| Eos resting | 0.33 |
| Eos IL-4 4 hr | 0.09 |
| Eos IL-4 24 hr | 0.87 |
| Macrophage-resting | 0.00 |
| Macrophage-LPS 4 hr | 0.00 |
| Macrophage-LPS 24 hr | 0.00 |
| Macrophage-IFNg 4 hr | 0.00 |
| Macrophage-IFNg 24 hr | 0.02 |
| Granulocytes resting | 1.39 |
| Granulocytes INFg 4 hr | 0.89 |
| Granulocytes INFg 24 hr | 0.26 |
| Granulocytes TNFa 4 hr | 1.49 |
| Granulocytes TNFa 24 hr | 0.67 |
| THO(RL)-0 | 0.24 |
| THO(RL)-1 | 1.16 |
| THO(RL)-6 | 0.23 |
| THO(RL)-24 | 0.11 |
| TH1(RL)-0 | 0.71 |
| TH1(RL)-1 | 1.89 |
| TH1(RL)-6 | 0.63 |
| TH1(RL)-24 | 0.17 |
| TH2(RL)-0 | 0.05 |
| TH2(RL)-1 | 1.74 |
| TH2(RL)-6 | 0.10 |
| TH2(RL)-24 | 0.06 |
| NHBE resting | 0.00 |
| NHBE IL-4 4 hr | 0.02 |
| NHBE IL-4 24 hr | 0.01 |
| NHBE IL-13 4 hr | 0.04 |
| NHBE IL-13 24 hr | 0.00 |
| NHBE IL-4/IL-13 4 hr | 0.00 |
| NHBE IL-4/IL-13 24 hr | 0.00 |

TABLE 4
Expression of PYRIN-9

| Cell Type | Relative Expression |
|---|---|
| BSMC resting | 0.03 |
| BSMC IL-1 4 hr | 0.00 |
| BSMC IL-1 24 hr | 0.01 |
| BSMC IFNg 4 hr | 0.00 |
| BSMC IFNg 24 hr | 0.00 |
| BSMC TNFa 4 hr | 0.00 |
| BSMC TNFa 24 hr | 0.00 |
| NHLF resting | 0.00 |
| NHLF TGFb 4 hr | 0.01 |
| NHLF TGFb 24 hr | 0.00 |
| NHLF TNFa 4 hr | 0.00 |
| NHLF TNFa 24 hr | 0.00 |
| NHDF resting | 0.07 |
| NHDF TGFb 4 hr | 0.00 |
| NHDF TNFa 4 hr | 0.01 |
| NHDF TNFa 24 hr | 0.00 |
| Jag 3,4,9 Bronchitis | 0.09 |
| JAG 1,2,5,7 Asthma | 0.04 |
| EUR 64 | 0.24 |
| AMC 362 | 0.03 |
| AMC 364 | 0.17 |
| AMC 365 | 0.07 |
| AMC 366 | 0.06 |
| N. Lung PIT 242 | 0.29 |
| N Lung CHT 427 | 0.05 |
| N Lung CHT 810 | 0.00 |
| N Lung CHT 894 | 0.06 |
| N Lung CHT 1242 | 0.12 |
| N Lung CHT 700 | 0.01 |
| N Lung CHT 702 | 0.07 |
| N Lung CHT 834 | 0.00 |
| N Lung MDA 180 | 0.06 |
| N Lung MDA 184 | 0.02 |
| N Lung MDA 185 | 0.01 |
| COPD MDA 177 | 0.15 |
| COPD NDR 187 | 1.08 |
| COPD NDR 188 | 0.06 |
| COPD MDA 189 | 0.04 |
| COPD CHT 743 | 1.01 |
| IPF B | 0.23 |
| IPF C | 1.11 |
| IPF D3 | 0.15 |
| IPF E4 | 4.78 |
| Norm. Liver NDR200 | 0.14 |
| NDR 141 | 0.29 |
| NDR 191 | 0.64 |

PYRIN-9 Activates CARD-5 Resulting in Increased NF-κB Activity

NF-κB is activated when high levels of CARD-5 are expressed in a cell. Experiments were carried out to determine whether PYRIN-9 synergizes with CARD-5 to activate NF-κB.

PYRIN-9 regulation of the NF-κB pathway is of interest because the NF-κB pathway is involved in many diseases (see, e.g., New England Journal of Medicine 336:1066 (1997) and American Journal of Cardiology 76:18C (1995)). Participation of PYRIN-9 in the NF-κB pathway makes PYRIN-9 an attractive target for drugs that modulate the NF-κB pathway for treatment of NF-κB pathway-dependent diseases, conditions, and biological processes.

The role of PYRIN-9 in NF-κB signaling pathways was examined using a luciferase reporter plasmid described previously (Wang et al. (2001) J. Biol. Chem. 276:2140509). 293T cells were transfected with pNF-κB luciferase reporter (Stratagene, La Jolla, Calif.), pRL-TK (Renilla luciferase reporter vector, Promega Corp., Madison, Wis.), and indicated expression plasmids. Cells were harvested and firefly and Renilla luciferase activities were determined using the DUAL-LUCIFERASE REPORTER (assay system using firefly and Renilla luciferase reporter activities in one sample, Promega Corp., Madison, Wis.).

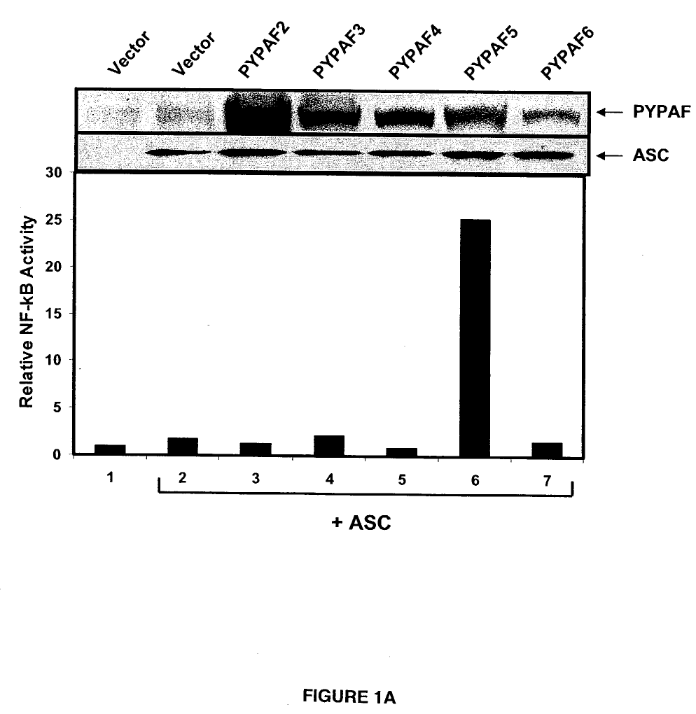

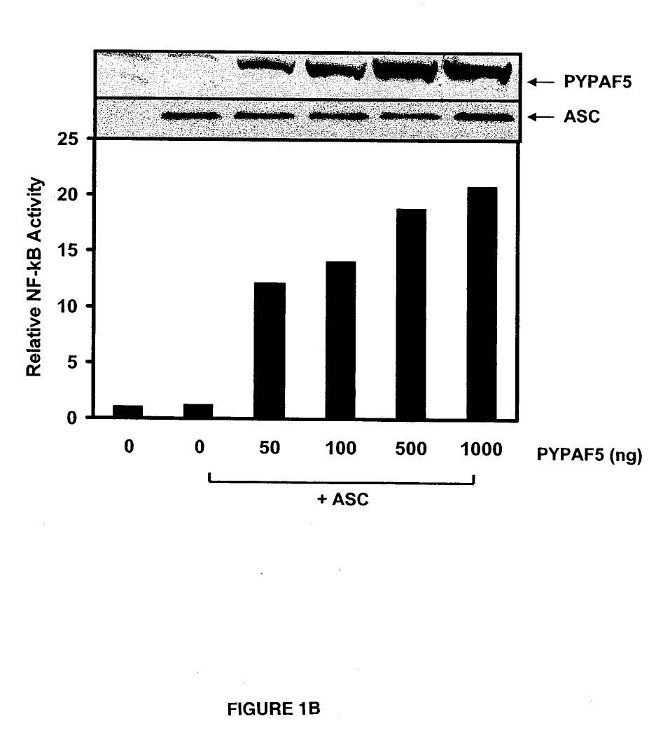

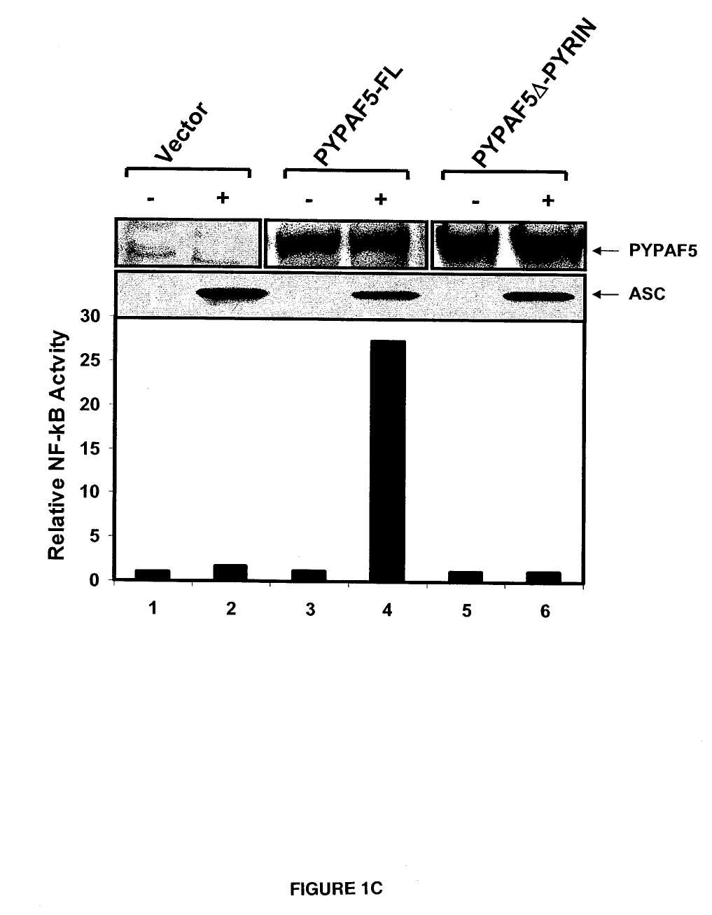

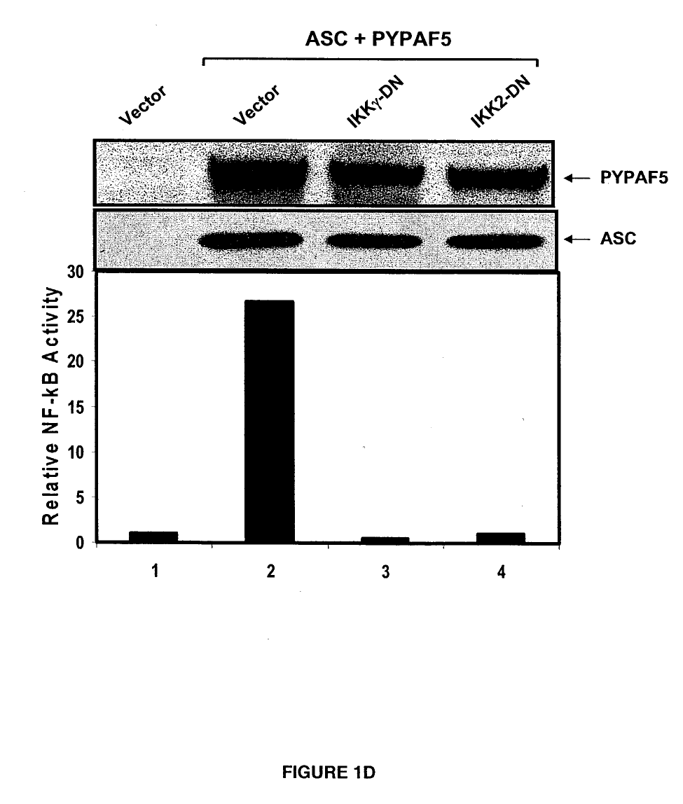

Expression of PYRIN-9 alone in cells failed to activate NF-κB. In contrast, NF-κB was activated when high levels of CARD-5 were expressed in cells. However, when CARD-5 was expressed at low protein levels that did not activate NF-κB, PYRIN-9 activated NF-κB (FIG. 1A, lane 6). PYRIN-9 and CARD-5 activated NF-κB in a concentration-dependent manner (FIG. 1B). The N-terminal PYRIN domain of PYRIN-9 was essential for NF-κB signaling, since deletion of this domain (PYPAF5-PYRIN) eliminated the synergistic induction of NF-κB activity (FIG. 1C, compare lanes 4 and 6). Immunoblot analysis revealed that CARD-5 protein levels were not increased when co-expressed with PYPAF5, demonstrating that the activation of NF-κB was not due to increased levels of CARD-5 (FIG. 1B, upper panels). NF-κB signaling occurred through the IKK complex because dominant-negative versions of IKK-y and IKK-2 blocked the ability of PYRIN-9 to synergistically activate NF-κB (FIG. 1D, lanes 3 and 4). These data demonstrate that PYRIN-9 is an activator of NF-κB signaling when co-expressed with CARD-5.

PYRIN-9 and CARD-5 Synergistically Activate Caspase-1

CARD-5 is a relatively small protein containing an N-terminal PYRIN domain and a C-terminal CARD domain (see, e.g., Masumoto et al. (1999) J. Biol. Chem. 274:33835-38). Experiments were carried out using an IL-1β secretion assay to determine whether CARD-5 and PYRIN-9 induce activation of caspase-1. Active caspase-1 cleaves pro-IL-1β resulting in the secretion of bioactive IL-1β from cells. In the IL-1β secretion assays, COS-7L cells (GIBCO) were co-transfected in 12-well (22-mm) plates using LIPOFECTAMINE 2000 transfection reagent (Invitrogen Life Technologies, Carlsbad, Calif.) with plasmids encoding mouse pro-IL-1β and indicated expression plasmids (total DNA=1.04 μg). Supernatants were collected 24 hours after transfection and subjected to ELISA for mouse IL-1β according to the manufacturer's protocol (R&D Systems, Minneapolis, Minn.).

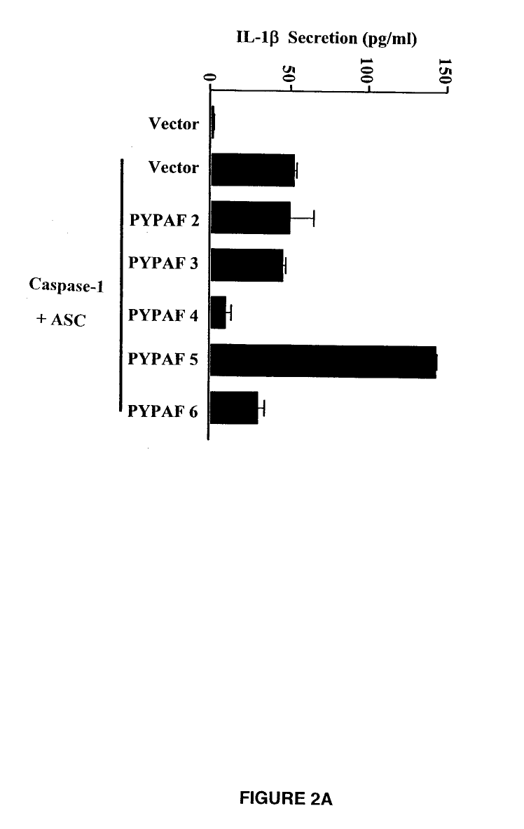

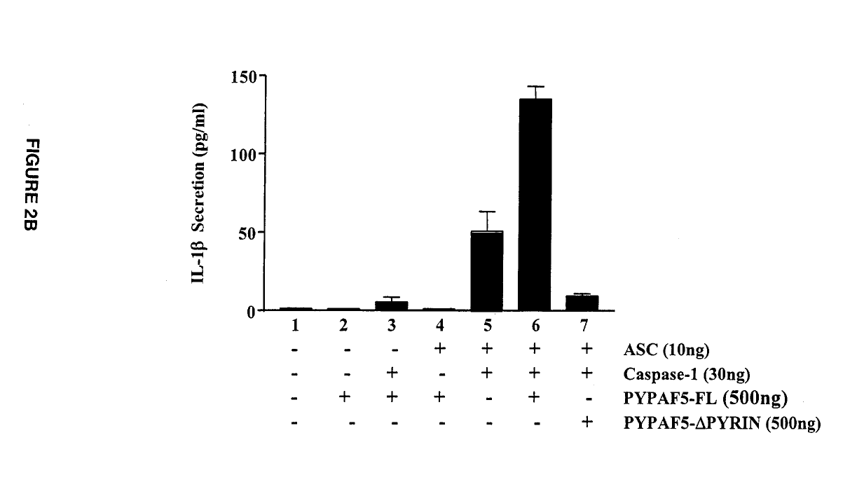

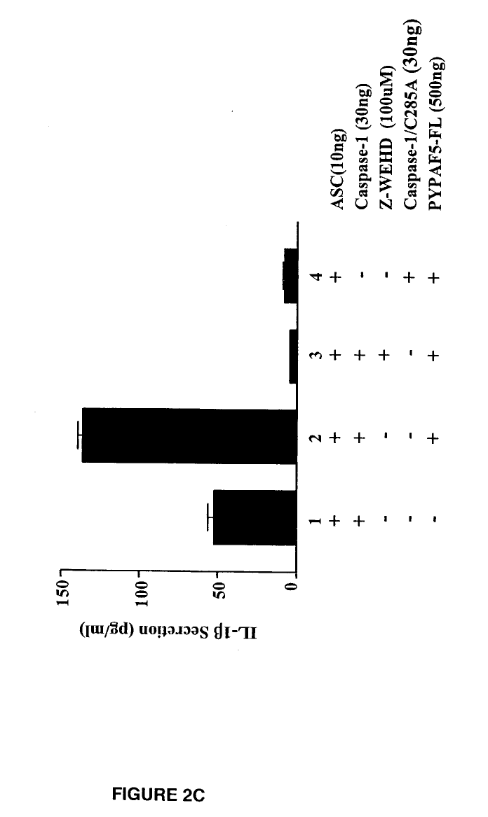

When CARD-5 was expressed at protein levels that resulted in low levels of caspase activity, co-expression with PYRIN-9 resulted in a synergistic activation of pro-caspase-1 and a corresponding increase in IL-1β production (FIG. 2A). The N-terminal PYRIN domain of PYRIN-9 was essential for pro-caspase-1 activation, since deletion of this domain (PYRIN-9-PYRIN) eliminated the synergistic production of IL-1β (FIG. 2B). Immunoblot analysis revealed that PYRIN-9-PYRIN was expressed at levels similar to that of PYRIN-9, indicating that loss of function was not due to reduced protein levels. PYRIN-9 and CARD-5 were unable to synergistically induce cytokine expression when co-expressed with a caspase-1 active site mutant (C285A) (FIG. 2C). Moreover, addition of a caspase-1 inhibitor (z-WEHD-FMK, R&D Systems, Minneapolis, Minn.) blocked the ability of PYRIN-9 and CARD-5 to induce the secretion of IL-1β (FIG. 2C). Taken together, these data demonstrate that PYRIN-9 and CARD-5 synergistically activate pro-caspase-1.

Plasmids expressing mouse pro-IL-1β with a C-terminal FLAG epitope or human pro-caspase-1 with a C-terminal Myc epitope were constructed using pCI mammalian expression vector (Promega, Madison, Wis.). Plasmids expressing either dominant negative IKKγ or inactive pro-caspase-1 (C285) were described previously (Geddes et al. (2001) Biochem. Biophys. Res. Commun. 284:77-82; Manji et al. (2001) J. Biol. Chem. 277:11570-75), the teachings of which are incorporated herein by this reference.

CARD-5 has previously been shown to interact with caspase-1 and induce apoptosis (see, e.g., U.S. application Ser. No. 09/841,879, filed Apr. 24, 2001, and U.S. application Ser. No. 09/728,721, filed Dec. 1, 2000, the contents of which are incorporated by reference). Thus, based upon the findings presented herein, PYRIN-9 is also expected to participate in apoptotic and/or inflammatory signaling pathways.

PYRIN-9 is a member of a family of molecules having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics.

Preferred PYRIN-9 polypeptides of the present invention include an amino acid sequence sufficiently identical to one or more of the following domains: a pyrin domain, and NBS domain, and/or a LRR domain.

As used interchangeably herein a “PYRIN-9 activity”, “biological activity of PYRIN-9” or “functional activity of PYRIN-9”, refers to an activity exerted by a PYRIN-9 protein, polypeptide or nucleic acid molecule on a PYRIN-9 responsive cell as determined in vivo, or in vitro, according to standard techniques. PYRIN-9 may act as a pro-apoptotic protein or an anti-apoptotic protein (i.e., it might act to decrease or increase apoptosis). A PYRIN-9 activity can be a direct activity, such as an association with or an enzymatic activity on a second protein or an indirect activity, such as a cellular signaling activity mediated by interaction of the PYRIN-9 protein with a second protein.

In one embodiment, a PYRIN-9 activity can include at least one or more of the following activities: (i) the ability to interact with proteins in an apoptotic or inflammatory signaling pathway, e.g., CARD-5 and/or caspase-1; (ii) the ability to interact with a PYRIN-9; (iii) the ability to bind to and/or hydrolyze a nucleotide, e.g., ATP or GTP; (iv) the ability to interact with an intracellular target protein; (v) the ability to interact, directly or indirectly, with one or more proteins having a pyrin domain, a CARD domain, or other domain associated with apoptotic and/or inflammatory signaling; (vi) the ability to modulate, directly or indirectly, the activity of a caspase, e.g., caspase-1 or caspase-9; (vii) the ability to induce the activity of caspase-1; (viii) the ability to modulate of ER-specific apoptosis pathways; (ix) the ability to modulate (increase or decrease), directly or indirectly, the activity of NF-κB; (x) the ability to increase the activity of NF-κB; (xi) the ability to modulate, directly or indirectly, Apaf-1; (xii) the ability to modulate apoptosis and/or inflammation; (xiii) the ability to interact, directly or indirectly, with a Bcl-2 family member; (xiv) the ability to modulate, directly or indirectly, the activity of a stress activated kinase (e.g., JNK/p38); and (xv) the ability to modulate, directly or indirectly, phosphorylation of CHOP (GADD 153). PYRIN-9 nucleic acids and polypeptides as well as modulators of activity or expression of PYRIN-9 might be used to modulate an Apaf-1 signaling pathway.

Accordingly, another embodiment of the invention features isolated PYRIN-9 proteins and polypeptides having a PYRIN-9 activity.

Various aspects of the invention are described in further detail in the following subsections.

Isolated Nucleic Acid Molecules

One aspect of the invention pertains to isolated nucleic acid molecules that encode PYRIN-9 proteins or biologically active portions thereof, as well as nucleic acid molecules sufficient for use as hybridization probes to identify PYRIN-9-encoding nucleic acids (e.g., PYRIN-9 mRNA) and fragments for use as PCR primers for the amplification or mutation of PYRIN-9 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) that which naturally flank the nucleic acid (i.e., sequences located at the 5′ and 3′ ends of the nucleic acid) in the genomic DNA of the organism from which the nucleic acid is derived. For example, in various embodiments, the isolated PYRIN-9 nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, a domain-encoding portion thereof or a complement of these nucleotide sequences, can be isolated using standard molecular biology techniques and the sequence information provided herein. Using all or portion of the nucleic acid sequences of SEQ ID NO:1 as a hybridization probe, PYRIN-9 nucleic acid molecules can be isolated using standard hybridization and cloning techniques (e.g., as described in Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

A nucleic acid of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to PYRIN-9 nucleotide sequences can be prepared by standard synthetic techniques, e.g., using an automated DNA synthesizer.

In another embodiment, an isolated nucleic acid molecule of the invention comprises a nucleic acid molecule which is a complement of the nucleotide sequence shown in SEQ ID NO:1, or a portion thereof. A nucleic acid molecule which is complementary to a given nucleotide sequence is one which is sufficiently complementary to the given nucleotide-sequence that it can hybridize to the given nucleotide sequence thereby forming a stable duplex.

Moreover, the nucleic acid molecule of the invention can comprise only a portion of a nucleic acid sequence encoding PYRIN-9, for example, a fragment which can be used as a probe or primer or a fragment encoding a biologically active portion of PYRIN-9. The nucleotide sequence determined from the cloning of the PYRIN-9 gene allows for the generation of probes and primers designed for use in identifying and/or cloning PYRIN-9 homologues in other cell types, e.g., from other tissues, as well as PYRIN-9 homologues and orthologs from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350, 400, 500, 1000, or more consecutive nucleotides of the sense or anti-sense sequence of SEQ ID NO:1, or of a naturally occurring mutant of one of SEQ ID NO:1.

Probes based on the PYRIN-9 nucleotide sequence can be used to detect transcripts or genomic sequences encoding the same or similar proteins. The probe comprises a label group attached thereto, e.g., a radioisotope, a fluorescent compound, an enzyme, or an enzyme co-factor. Such probes can be used as a part of a diagnostic test kit for identifying allelic variants and orthologs of the PYRIN-9 proteins of the present invention, identifying cells or tissue which mis-express a PYRIN-9 protein, such as by measuring a level of a PYRIN-9-encoding nucleic acid in a sample of cells from a subject, e.g., detecting PYRIN-9 mRNA levels or determining whether a genomic PYRIN-9 gene has been mutated or deleted.

Another embodiment includes a fragment which includes a nucleotide sequence encoding an amino acid fragment described herein. Nucleic acid fragments can encode a specific domain or site described herein or fragments thereof, particularly fragments thereof which are at least 25, 50 or 75 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a PYRIN-9 nucleic acid fragment can include a sequence corresponding to a pyrin domain, an NBS domain, a kinase 1a domain, a Motif II, a kinase 2 domain, a kinase 3a domain, a Motif V, a Motif VI, a Motif VII, a LRR domain, or a leucine rich repeat a, b, c, or d as described herein.

A nucleic acid fragment encoding a “biologically active portion” of PYRIN-9 can be prepared by isolating a portion of SEQ ID NO:1 which encodes a polypeptide having a PYRIN-9 biological activity, expressing the encoded portion of PYRIN-9 protein (e.g., by recombinant expression in vitro) and assessing the activity of the encoded portion of PYRIN-9.

The invention further encompasses nucleic acid molecules that differ from the nucleotide sequence of SEQ ID NO:1 due to degeneracy of the genetic code and thus encode the same PYRIN-9 protein as that encoded by the nucleotide sequence shown in SEQ ID NO:1.

In addition to the PYRIN-9 nucleotide sequences shown in SEQ ID NO:1, it will be appreciated by those skilled in the art that DNA sequence polymorphisms that lead to changes in the amino acid sequences of PYRIN-9 may exist within a population (e.g., the human population). Such genetic polymorphism in the PYRIN-9 gene may exist among individuals within a population due to natural allelic variation. As used herein, the terms “gene” and “recombinant gene” refer to nucleic acid molecules comprising an open reading frame encoding a PYRIN-9 protein, preferably a mammalian PYRIN-9 protein. Such natural allelic variations can typically result in 1-5% variance in the nucleotide sequence of the PYRIN-9 gene. Any and all such nucleotide variations and resulting amino acid polymorphisms in PYRIN-9 that are the result of natural allelic variation and that do not alter the functional activity of PYRIN-9 are intended to be within the scope of the invention. Thus, e.g., 1%, 2%, 3%, 4%, or 5% of the amino acids in PYRIN-9 (e.g., 1, 2, 3, 4, 5, 6, 8, 10, 15, or 17 amino acids) are replaced by another amino acid, preferably by conservative substitution.

Moreover, nucleic acid molecules encoding PYRIN-9 proteins from other species (PYRIN-9 orthologs/homologues), which have a nucleotide sequence which differs from that of a PYRIN-9 disclosed herein, are intended to be within the scope of the invention.

Accordingly, in another embodiment, an isolated nucleic acid molecule of the invention is at least 150 (225, 300, 325, 350, 375, 400, 425, 450, 500, 550, 600, 650, 700, 800, 900, 1000, 1100, 1200, 1300, 1400, 1500, 1600, 1800, 2000, 2250, 2500, or 2600) nucleotides in length and hybridizes under stringent conditions to the nucleic acid molecule comprising the nucleotide sequence, preferably the coding sequence, of SEQ ID NO:1.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. An, non-limiting example of stringent hybridization conditions are hybridization in 6× sodium chloride/sodium citrate (SSC) at about 45° C., followed by one or more washes in 0.2×SSC, 0.1% SDS at 50-65° C. (e.g., 50° C. or 60° C. or 65° C.). Preferably, the isolated nucleic acid molecule of the invention that hybridizes under stringent conditions corresponds to a naturally-occurring nucleic acid molecule. As used herein, a “naturally-occurring” nucleic acid molecule refers to an RNA or DNA molecule having a nucleotide sequence that occurs in a human cell in nature (e.g., encodes a natural protein).

In addition to naturally-occurring allelic variants of the PYRIN-9 sequence that may exist in the population, the skilled artisan will further appreciate that changes can be introduced by mutation into the nucleotide sequence of SEQ ID NO:1, thereby leading to changes in the amino acid sequence of the encoded protein without altering the functional ability of the protein. For example, one can make nucleotide substitutions leading to amino acid substitutions at “non-essential” amino acid residues. A “non-essential” amino acid residue is a residue that can be altered from the wild-type sequence of PYRIN-9 protein without altering the biological activity, whereas an “essential” amino acid residue is required for biological activity. For example, amino acid residues that are conserved among the PYRIN-9, proteins of various species are predicted to be particularly unamenable to alteration.

For example, preferred PYRIN-9 proteins of the present invention contain at least one domain identified herein, e.g. a pyrin domain, an NBS domain or a LRR domain. Such conserved domains are less likely to be amenable to mutation. Other amino acid residues, however, (e.g., those that are not conserved or only semi-conserved among PYRIN-9 of various species) may not be essential for activity and thus are likely to be amenable to alteration.

Accordingly, another aspect of the invention pertains to nucleic acid molecules encoding PYRIN-9 proteins that contain changes in amino acid residues that are not essential for activity. Such PYRIN-9 proteins differ in amino acid sequence from SEQ ID NO:2 and yet retain biological activity. In one embodiment, the isolated nucleic acid molecule includes a nucleotide sequence encoding a protein that includes an amino acid sequence that is at least about 45% identical, 65%, 75%, 85%, 95%, 98% or 99% identical to the amino acid sequence of SEQ ID NO:2. An isolated nucleic acid molecule encoding a PYRIN-9 protein having a sequence which differs from that of SEQ ID NO:1 can be created by introducing one or more nucleotide substitutions, additions or deletions into the nucleotide sequence of PYRIN-9 (SEQ ID NO:1) such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. Thus, for example, 1%, 2%, 3%, 5%, or 10% of the amino acids can be replaced by conservative substitution. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in PYRIN-9 is preferably replaced with another amino acid residue from the same side chain family. Alternatively, mutations can be introduced randomly along all or part of a PYRIN-9 coding sequence, such as by saturation mutagenesis, and the resultant mutants can be screened for PYRIN-9 biological activity to identify mutants that retain activity. Following mutagenesis, the encoded protein can be expressed recombinantly and the activity of the protein can be determined.

In an embodiment, a mutant PYRIN-9 protein can be assayed for: (1) the ability to form protein:protein interactions with at least one protein in the apoptotic signaling pathway; (2) the ability to bind a PYRIN-9 ligand; or (3) the ability to bind to an intracellular target protein.

The present invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire PYRIN-9 coding strand, or to only a portion thereof, e.g., all or part of the protein coding region (or open reading frame). An antisense nucleic acid molecule can be antisense to a noncoding region of the coding strand of a nucleotide sequence encoding PYRIN-9. The noncoding regions (“5” and 3“untranslated regions”) are the 5′ and 3′ sequences that flank the coding region and are not translated into amino acids. Given the coding strand sequences encoding PYRIN-9 disclosed herein, antisense nucleic acids of the invention can be designed according to the rules of Watson and Crick base pairing. The antisense nucleic acid molecule can be complementary to the entire coding region of PYRIN-9 mRNA, but more preferably is an oligonucleotide which is antisense to only a portion of the coding or noncoding region of PYRIN-9 mRNA. For example, the antisense oligonucleotide can be complementary to the region surrounding the translation start site of PYRIN-9 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-aino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a PYRIN-9 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An antisense nucleic acid molecule of the invention can be administered by direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave PYRIN-9 mRNA transcripts to thereby inhibit translation of PYRIN-9 mRNA. A ribozyme having specificity for a PYRIN-9-encoding nucleic acid can be designed based upon the nucleotide sequence of a PYRIN-9 cDNA disclosed herein. For example, a derivative of a Tetrahymena L-19 IVS RNA can be constructed in which the nucleotide sequence of the active site is complementary to the nucleotide sequence to be cleaved in a PYRIN-9-encoding mRNA. See, e.g., Cech et al. U.S. Pat. No. 4,987,071; and Cech et al. U.S. Pat. No. 5,116,742. Alternatively, PYRIN-9 mRNA can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, PYRIN-9 gene expression can be inhibited by targeting nucleotide sequences complementary to the regulatory region of the PYRIN-9 (e.g., the PYRIN-9 promoter and/or enhancers) to form triple helical structures that prevent transcription of the PYRIN-9 gene in target cells. See generally, Helene (1991) Anticancer Drug Des. 6(6):569-84; Helene (1992) Ann. N.Y. Acad. Sci. 660:27-36; and Maher (1992) Bioassays 14(12):807-15.

In embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93:14670-675.

PNAs of PYRIN-9 can be used for therapeutic and diagnostic applications. For example, PNAs can be used as antisense or antigene agents for sequence-specific modulation of gene expression by, e.g., inducing transcription or translation arrest or inhibiting replication. PNAs of PYRIN-9 can also be used, e.g., in the analysis of single base pair mutations in a gene by, e.g., PNA directed PCR clamping; as artificial restriction enzymes when used in combination with other enzymes, e.g., S1 nucleases (Hyrup (1996) supra; or as probes or primers for DNA sequence and hybridization (Hyrup (1996) supra; Perry-OKeefe et al. (1996) Proc. Natl. Acad. Sci. USA 93: 14670-675).

In another embodiment, PNAs of PYRIN-9 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of PYRIN-9 can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) supra). The synthesis of PNA-DNA chimeras can be performed as described in Hyrup (1996) supra and Finn et al. (1996) Nucleic Acids Research 24(17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag et al. (1989) Nucleic Acid Res. 17:5973-88). PNA monomers are then coupled in a stepwise manner to produce a chimeric molecule with a 5′ PNA segment and a 3′ DNA segment (Finn et al. (1996) Nucleic Acids Research 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

Isolated PYRIN-9 Proteins and Anti-PYRIN-9 Antibodies

One aspect of the invention pertains to isolated PYRIN-9 proteins, or fragments thereof, e.g., biologically active portions, for use, e.g., in screening assays, as therapeutic or diagnostic targets, or as immunogens or antigens to raise or test (or more generally to bind) anti-PYRIN-9 antibodies. In one embodiment, native PYRIN-9 proteins can be isolated from cells or tissue sources by an appropriate purification scheme using standard protein purification techniques. In another embodiment, PYRIN-9 proteins are produced by recombinant DNA techniques. Alternative to recombinant expression, a PYRIN-9 protein or polypeptide can be synthesized chemically using standard peptide synthesis techniques.

An “isolated” or “purified” protein or biologically active portion thereof is substantially free of cellular material or other contaminating proteins from the cell or tissue source from which the PYRIN-9 protein is derived, or substantially free from chemical precursors or other chemicals when chemically synthesized. The language “substantially free of cellular material” includes preparations of PYRIN-9 protein in which the protein is separated from cellular components of the cells from which it is isolated or recombinantly produced. Thus, PYRIN-9 protein that is substantially free of cellular material includes preparations of PYRIN-9 protein having less than about 30%, 20%, 10%, or 5% (by dry weight) of non-PYRIN-9 protein (also referred to herein as a “contaminating protein”). When the PYRIN-9 protein or biologically active portion thereof is recombinantly produced, it is also preferably substantially free of culture medium, i.e., culture medium represents less than about 20%, 10%, or 5% of the volume of the protein preparation. When PYRIN-9 protein is produced by chemical synthesis, it is preferably substantially free of chemical precursors or other chemicals, i.e., it is separated from chemical precursors or other chemicals which are involved in the synthesis of the protein. Accordingly such preparations of PYRIN-9 protein have less than about 30%, 20%, 10%, 5% (by dry weight) of chemical precursors or non-PYRIN-9 chemicals.

Biologically active portions of a PYRIN-9 protein include peptides comprising amino acid sequences sufficiently identical to or derived from the amino acid sequence of the PYRIN-9 protein (e.g., the amino acid sequence shown in SEQ ID NO:2), which include fewer amino acids than the full length PYRIN-9 protein, and exhibit at least one activity of a PYRIN-9 protein. Typically, biologically active portions comprise a domain or motif with at least one activity of the PYRIN-9 protein. A biologically active portion of a PYRIN-9 protein can be a polypeptide which is, for example, 10, 25, 50, 72, 75, 100, 125, 150, 175, 200, 225, 250, 272, 300, 325, 350, 375, 400, 425, 450, 500, 600, 700, or more amino acids in length. Preferred biologically active polypeptides include one or more identified PYRIN-9 structural domains or sequence motifs, e.g., a pyrin domain, an NBS domain, or a portion thereof, e.g. a kinase 1a domain, a Motif II, a kinase 2 domain, a kinase 3a domain, a Motif V, a Motif VI, a Motif VII, or a LRR domain, or or a portion thereof, e.g. a leucine rich repeat a, b, c, or d.

Moreover, other biologically active portions, in which other regions of the protein are deleted, can be prepared by recombinant techniques and evaluated for one or more of the functional activities of a native PYRIN-9 protein.

Human PYRIN-9 proteins have the amino acid sequences of SEQ ID NO:2. Other useful PYRIN-9 proteins are substantially identical to SEQ ID NO:2 and retain the functional activity of the protein of SEQ ID NO:2 yet differ in amino acid sequence due to natural allelic variation or mutagenesis.

In the context of an amino acid sequence, the terms “substantially identical” and “sufficiently identical” are used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2 are termed substantially identical or sufficiently identical.

A useful PYRIN-9 protein is a protein which includes an amino acid sequence at least about 45%, preferably 55%, 65%, 75%, 85%, 95%, 98% or 99% identical to the amino acid sequence of SEQ ID NO:2 and retains the functional activity of the PYRIN-9 protein of SEQ ID NO:2, e.g., the ability to activate caspase-1 and/or activate NF-κB.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences similar or homologous to PYRIN-9 nucleic acid molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used (accessible at the website maintained by National Center for Biotechnology Information, Bethesda, Md., USA (ncbi.nlm.nih.gov). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package (available at the bioinformatics page of the website maintained by Accelrys, Inc., San Diego, Calif., USA). When utilizing the ALIGN program for comparing amino acid sequences, a PAM120 weight residue table, a gap length penalty of 12, and a gap penalty of 4 can be used. When utilizing the ALIGN program for comparing nucleic acid sequences, a gap length penalty of 12, and a gap penalty of 4 can be used. Another preferred example of a mathematical algorithm utilized for the comparison of sequences is the Needleman and Wunsch (J. Mol. Biol. (48):444-453 (1970)) algorithm which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6.

The percent identity between two sequences can be determined using techniques similar to those described above, with or without allowing gaps. In calculating percent identity, typically exact matches are counted.

The invention also provides PYRIN-9 chimeric or fusion proteins. As used herein, a PYRIN-9 “chimeric protein” or “fusion protein” comprises a PYRIN-9 polypeptide, e.g. PYRIN-9 protein or a fragment thereof operatively linked to a non-PYRIN-9 polypeptide. A “PYRIN-9 polypeptide” refers to a polypeptide having an amino acid sequence corresponding to all or at least one portion (preferably a biologically active portion) of a PYRIN-9 protein, e.g., a pyrin domain, an NBS domain, or a portion thereof, e.g. a kinase 1a domain, a Motif II, a kinase 2 domain, a kinase 3a domain, a Motif V, a Motif VI, a Motif VII, or a LRR domain, or or a portion thereof, e.g. a leucine rich repeat a, b, c, or d, whereas a “non-PYRIN-9 polypeptide” refers to a polypeptide having an amino acid sequence corresponding to a protein which is not substantially identical to the PYRIN-9 protein, e.g., a protein which is different from the PYRIN-9 proteins and which is derived from the same or a different organism. Within the fusion protein, the term “operatively linked” is intended to indicate that the PYRIN-9 polypeptide and the non-PYRIN-9 polypeptide are fused in-frame to each other. The heterologous polypeptide can be fused to the N-terminus or C-terminus of the PYRIN-9 polypeptide.

In one embodiment, the fusion protein comprises a detectable label (e.g., an enzyme label (glutathione-S-transferase (GST)), fluorescent protein (e.g., Green Fluorescent Protein (GFP)), an antigen or epitope tag (e.g., hemagglutinin (HA), FLAG peptide, myc epitope tag), a binding domain) as the non-PYRIN-9 polypeptide, optionally joined to the PYRIN-9 polypeptide via a (one or more) linker sequence. Additional (e.g., third, fourth) moieties can be present as appropriate. In another embodiment, the fusion protein comprises an affinity label, e.g. a tag (e.g. a polyhistidine tag which confers binding to a chromatography medium with chelated metal (e.g., nickel)) as the non-PYRIN-9 polypeptide. One useful fusion protein is a GST fusion protein in which the PYRIN-9 sequences are fused to the C-terminus of the GST sequences. Such fusion proteins can facilitate the purification of recombinant PYRIN-9. In another embodiment, the fusion protein contains a signal sequence from another protein. In certain host cells (e.g., mammalian host cells), expression and/or secretion of PYRIN-9 can be increased through use of a heterologous signal sequence. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Molecular cloning, Sambrook et al, second edition, Cold spring harbor laboratory press, 1989) and the protein A secretory signal (Pharmacia Biotech; Piscataway, N.J.).

In yet another embodiment, the fusion protein is a PYRIN-9-immunoglobulin fusion protein in which all or part of PYRIN-9 is fused to sequences derived from a member of the immunoglobulin protein family e.g., a portion of an immunoglobulin (e.g., IgG, IgA, or IgE), e.g., an Fc region and/or the hinge C1 and C2 sequences of an immunoglobulin. The PYRIN-9-immunoglobulin fusion proteins of the invention can be incorporated into pharmaceutical compositions and administered to a subject to inhibit an interaction between a PYRIN-9 ligand and a PYRIN-9 protein on the surface of a cell, to thereby suppress PYRIN-9-mediated signal transduction in vivo. The PYRIN-9-immunoglobulin fusion proteins can be used to affect the bioavailability of a PYRIN-9 cognate ligand. Inhibition of the PYRIN-9 ligand/PYRIN-9 interaction may be useful therapeutically for both the treatment of proliferative and differentiative disorders, as well as modulating (e.g., promoting or inhibiting) cell survival. Moreover, the PYRIN-9-immunoglobulin fusion proteins of the invention can be used as immunogens to produce anti-PYRIN-9 antibodies in a subject, to purify PYRIN-9 ligands and in screening assays to identify molecules which inhibit the interaction of PYRIN-9 with a PYRIN-9 ligand.

Preferably, a PYRIN-9 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A PYRIN-9-encoding nucleic acid can be cloned into such an expression vector such that the fusion moiety is linked in-frame to the PYRIN-9 protein.

The present invention also pertains to variants of the PYRIN-9 proteins which function as either PYRIN-9 agonists (mimetics) or as PYRIN-9 antagonists. Variants of the PYRIN-9 proteins can be generated by mutagenesis, e.g., discrete point mutation or truncation of the PYRIN-9 proteins. An agonist of the PYRIN-9 protein can retain substantially the same, or a subset, of the biological activities of the naturally occurring form of the PYRIN-9 protein. An antagonist of the PYRIN-9 protein can inhibit one or more of the activities of the naturally occurring form of the PYRIN-9 protein by, for example, competitively binding to a downstream or upstream member of a cellular signaling cascade which includes the PYRIN-9 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the PYRIN-9 proteins.

Variants of the PYRIN-9 protein which function as either PYRIN-9 agonists (mimetics) or as PYRIN-9 antagonists can be identified by screening combinatorial libraries of mutants, e.g., truncation mutants of the PYRIN-9 protein for PYRIN-9 protein agonist or antagonist activity. In one embodiment, a variegated library of PYRIN-9 variants is generated by combinatorial mutagenesis at the nucleic acid level and is encoded by a variegated gene library. A variegated library of PYRIN-9 variants can be produced by, for example, enzymatically ligating a mixture of synthetic oligonucleotides into gene sequences such that a degenerate set of potential PYRIN-9 sequences is expressible as individual polypeptides, or alternatively, as a set of larger fusion proteins (e.g., for phage display) containing the set of PYRIN-9 sequences therein. There are a variety of methods which can be used to produce libraries of potential PYRIN-9 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PYRIN-9 sequences. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

Useful fragments of PYRIN-9, include fragments comprising or consisting of a domain or subdomain described herein, e.g., a pyrin domain, an NBS domain, or a portion thereof, e.g. a kinase 1a domain, a Motif II, a kinase 2 domain, a kinase 3a domain, a Motif V, a Motif VI, a Motif VII, or a LRR domain, or or a portion thereof, e.g. a leucine rich repeat a, b, c, or d.

In addition, libraries of fragments of the PYRIN-9 protein coding sequence can be used to generate a variegated population of PYRIN-9 fragments for screening and subsequent selection of variants of a PYRIN-9 protein. In one embodiment, a library of coding sequence fragments can be generated by treating a double stranded PCR fragment of a PYRIN-9 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the PYRIN-9 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PYRIN-9 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify PYRIN-9 variants (Arkin and Yourvan (1992) Proc. Natl. Acad. Sci. USA 89:7811-7815; Delgrave et al. (1993) Protein Engineering 6(3):327-331).

An isolated PYRIN-9 protein, or a portion or fragment thereof, can be used as an immunogen to generate antibodies that bind PYRIN-9 using standard techniques for polyclonal and monoclonal antibody preparation. The full-length PYRIN-9 protein can be used or, alternatively, the invention provides antigenic peptide fragments of PYRIN-9 for use as immunogens. The antigenic peptide of PYRIN-9 comprises at least 8 (preferably 10, 15, 20, or 30) amino acid residues of the amino acid sequence shown in SEQ ID NO:2 and encompasses an epitope of PYRIN-9 such that an antibody raised against the peptide forms a specific immune complex with PYRIN-9.

Useful antibodies include antibodies which bind to a domain or subdomain of PYRIN-9 described herein (e.g., a pyrin domain, an NBS domain, or a portion thereof, e.g. a kinase 1a domain, a Motif II, a kinase 2 domain, a kinase 3a domain, a Motif V, a Motif VI, a Motif VII, or a LRR domain, or or a portion thereof, e.g. a leucine rich repeat a, b, c, or d).

Preferred epitopes encompassed by the antigenic peptide are regions of PYRIN-9 that are located on the surface of the protein, e.g., hydrophilic regions. Other important criteria include a preference for a terminal sequence, high antigenic index (e.g., as predicted by Jameson-Wolf algorithm), ease of peptide synthesis (e.g., avoidance of prolines); and high surface probability (e.g., as predicted by the Emini algorithm).

A PYRIN-9 immunogen typically is used to prepare antibodies by immunizing a suitable subject, (e.g., rabbit, goat, mouse or other mammal) with the immunogen. An appropriate immunogenic preparation can contain, for example, recombinantly expressed PYRIN-9 protein or a chemically synthesized PYRIN-9 polypeptide. The preparation can further include an adjuvant, such as Freund's complete or incomplete adjuvant, or similar immunostimulatory agent. Immunization of a suitable subject with an immunogenic PYRIN-9 preparation induces a polyclonal anti-PYRIN-9 antibody response.

Accordingly, another aspect of the invention pertains to anti-PYRIN-9 antibodies. The term “antibody” as used herein refers to immunoglobulin molecules and immunologically active portions of immunoglobulin molecules, i.e., molecules that contain an antigen binding site which specifically binds an antigen, such as PYRIN-9. A molecule which specifically binds to PYRIN-9 is a molecule which binds PYRIN-9, but does not substantially bind other molecules in a sample, e.g., a biological sample, which naturally contains PYRIN-9. Examples of immunologically active portions of immunoglobulin molecules include F(ab) and F(ab′)2 fragments which can be generated by treating the antibody with an enzyme such as pepsin. The invention provides polyclonal and monoclonal antibodies that bind PYRIN-9. The term “monoclonal antibody” or “monoclonal antibody composition”, as used herein, refers to a population of antibody molecules that contain only one species of an antigen binding site capable of immunoreacting with a particular epitope of PYRIN-9. A monoclonal antibody composition thus typically displays a single binding affinity for a particular PYRIN-9 protein with which it immunoreacts.

Polyclonal anti-PYRIN-9 antibodies can be prepared as described above by immunizing a suitable subject with a PYRIN-9 immunogen. The anti-PYRIN-9 antibody titer in the immunized subject can be monitored over time by standard techniques, such as with an enzyme linked immunosorbent assay (ELISA) using immobilized PYRIN-9. If desired, the antibody molecules directed against PYRIN-9 can be isolated from the mammal (e.g., from the blood) and further purified by well-known techniques, such as protein A chromatography to obtain the IgG fraction. At an appropriate time after immunization, e.g., when the anti-PYRIN-9 antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a PYRIN-9 immunogen as described above, and the culture supernatants of the resulting hybridoma cells are screened to identify a hybridoma producing a monoclonal antibody that binds PYRIN-9.

Any of the many well known protocols used for fusing lymphocytes and immortalized cell lines can be applied for the purpose of generating an anti-PYRIN-9 monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from American Type Culture Collection (ATCC, Manassas, Va.). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind PYRIN-9, e.g., using a standard ELISA assay.

Alternative to preparing monoclonal antibody-secreting hybridomas, a monoclonal anti-PYRIN-9 antibody can be identified and isolated by screening a recombinant combinatorial immunoglobulin library (e.g., an antibody phage display library) with PYRIN-9 to thereby isolate immunoglobulin library members that bind PYRIN-9. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-PYRIN-9 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-PYRIN-9 antibody (e.g., monoclonal antibody) can be used to isolate PYRIN-9 by standard techniques, such as affinity chromatography or immunoprecipitation. An anti-PYRIN-9 antibody can facilitate the purification of natural PYRIN-9 from cells and of recombinantly produced PYRIN-9 expressed in host cells. Moreover, an anti-PYRIN-9 antibody can be used to detect PYRIN-9 protein (e.g., in a cellular lysate or cell supernatant) in order to evaluate the abundance and pattern of expression of the PYRIN-9 protein. Anti-PYRIN-9 antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator; or, biological response modifiers such as, for example, lymphokines, interleukin-1 (IL-1), interleukin-2 (IL-2), interleukin-6 (IL-6), granulocyte macrophase colony stimulating factor (GM-CSF), granulocyte colony stimulating factor (G-CSF), or other growth factors.

Techniques for conjugating such therapeutic moiety to antibodies are well known, see, e.g., Amon et al., “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al., “Antibodies for Drug Delivery”, in Controlled Drug Delivery (2nd Ed.), Robinson et al. (eds.), pp. 623-53 (Marcel Dekker, Inc. 1987); Thorpe, “Antibody Carriers of Cytotoxic Agents in Cancer Therapy: A Review”, in Monoclonal Antibodies '84: Biological and Clinical Applications, Pinchera et al. (eds.), pp. 475-506 (1985); “Analysis, Results, and Future Prospective of The Therapeutic Use of Radiolabeled Antibody In Cancer Therapy”, in Monoclonal Antibodies for Cancer Detection and Therapy, Baldwin et al. (eds.), pp. 303-16 (Academic Press 1985), and Thorpe et al., “The Preparation and Cytotoxic Properties of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

In addition, antibodies of the invention, either conjugated or not conjugated to a therapeutic moiety, can be administered together or in combination with a therapeutic moiety such as a cytotoxin, a therapeutic agent or a radioactive metal ion. The order of administration of the antibody and therapeutic moiety can vary. For example, in some embodiments, the antibody is administered concurrently (through the same or different delivery devices, e.g., syringes) with the therapeutic moiety. Alternatively, the antibody can be administered separately and prior to the therapeutic moiety. Still alternatively, the therapeutic moiety is administered separately and prior to the antibody. In many embodiments, these administration regimens will be continued for days, months or years.

Another aspect of the invention relates to a method for inducing an immunological response in a mammal which comprises inoculating the mammal with a PYRIN-9 polypeptide, adequate to produce antibody and/or T cell immune response to protect the animal from the diseases hereinbefore mentioned, amongst others. Yet another aspect of the invention relates to a method of inducing immunological response in a mammal which comprises, delivering a PYRIN-9 polypeptide via a vector directing expression of the polynucleotide and coding for the polypeptide in vivo in order to induce such an immunological response to produce antibody to protect the animal from diseases.

A further aspect of the invention relates to an immunological/vaccine formulation (composition) which, when introduced into a mammalian host, induces an immunological response in that mammal to a PYRIN-9 polypeptide of the present invention wherein the composition comprises a polypeptide or polynucleotide of PYRIN-9. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exist in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. This skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a work processing test file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or a target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration formed upon the folding of the target motif. There are a variety of target motifs know in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of know algorithms are disclosed publicly and a variety of commercially available software for conducting search means are and can be used in the computer-based systems of the present invention. Examples of such software include, but is not limited to, MacPattern (EMBL), BLASTIN and BLASTX (NCBIA).

For example, software that implements the BLAST (Altschul et al. (1990) J. of Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein-encoding fragments and are useful in producing commercially important proteins such as enzymes used in various reactions and in the production of commercially useful metabolites.

Recombinant Expression Vectors and Host Cells

Another aspect of the invention pertains to vectors, preferably expression vectors, containing a nucleic acid encoding PYRIN-9 (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., PYRIN-9 proteins, mutant forms of PYRIN-9, fusion proteins, etc.).

The recombinant expression vectors of the invention can be designed for expression of PYRIN-9 in prokaryotic or eukaryotic cells, e.g., bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident e prophage harboring a T7 gn1 gene under the transcriptional control of the lacUV5 promoter.

One strategy to maximize recombinant protein expression in E. coli is to express the protein in a bacterial having an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the PYRIN-9 expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), pGBT9 (Clontech, Palo Alto, Calif.), pGAD10 (Clontech, Palo Alto, Calif.), pYADE4 and pYGAE2 and pYPGE2 (Brunelli and Pall, (1993) Yeast 9:1299-1308), pYPGE15 (Brunelli and Pall, (1993) Yeast 9:1309-1318), pACTII (Dr. S. E. Elledge, Baylor College of Medicine), and picZ (InVitrogen Corp, San Diego, Calif.). Alternatively, PYRIN-9 can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840), pCI (Promega), and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. (supra). In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to PYRIN-9 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention or isolated nucleic acid molecule of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, PYRIN-9 protein can be expressed in bacterial cells such as E. coli, insect cells, yeast or mammalian cells (such as Chinese hamster ovary cells (CHO) or COS cells). Other suitable host cells are known to those skilled in the art.

Vector DNA or an isolated nucleic acid molecule of the invention can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In some cases vector DNA is retained by the host cell. In other cases the host cell does not retain vector DNA and retains only an isolated nucleic acid molecule of the invention carried by the vector. In some cases, and isolated nucleic acid molecule of the invention is used to transform a cell without the use of a vector.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding PYRIN-9 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a PYRIN-9 protein. Accordingly, the invention further provides methods for producing PYRIN-9 protein using the host cells of the invention. In one embodiment, the method comprises culturing the host cell of the invention (into which a recombinant expression vector or isolated nucleic acid molecule encoding PYRIN-9 has been introduced) in a suitable medium such that PYRIN-9 protein is produced. In another embodiment, the method further comprises isolating PYRIN-9 from the medium or the host cell.

PYRIN-9 nucleic acid molecules can be used in viral gene delivery systems for gene therapy, e.g., adenoviral or retroviral gene delivery systems.

PYRIN-9 nucleic acid molecules can also be used in non-viral gene delivery systems for gene therapy. Thus, another aspect of the invention pertains to non-viral gene delivery systems, such as plasmid-based gene delivery systems. Non-viral gene delivery systems are described in detail by Huang et al. ((1999) Nonviral Vectors for Gene Therapy, Academic Press, San Diego, Calif.). Nonviral vectors have several potential advantages over their viral counterparts, including: reduced immunogenicity; low acute toxicity; simplicity; and ease of large scale production. Nonviral vectors can be delivered as naked DNA, by bioballistic bombardment, and in various complexes, including liposome/DNA complexes (lipoplexes), polymer/DNA complexes (polyplexes), and liposome/polymer/DNA complexes (lipopolyplexes). Nonviral vectors may be administered by various routes, e.g., intravenous injection, peritoneal injection, intramuscular injection, subcutaneous injection, intratracheal injection, and aerosolization.

Naked DNA (i.e. free from association with, e.g., transfection-facilitating proteins, viral particles, liposomal formulations, charged lipids and calcium phosphate precipitating), can be expressed at its injection site or at a remote site. For example, naked DNA can be injected directly into skeletal muscle, liver, heart muscle, and tumor tissue. For systemic administration, plasmid DNA may need to be protected from degradation by endonucleases during delivery from the site of administration to the site of gene expression.

Bioballistic bombardment, also known as gene gun, allows for the penetration of target cells in vitro, ex vivo, or in vivo. In this technique, DNA-coated gold particles are accelerated to a high velocity by an electric arc generated by a high voltage discharge. The method is effective for a variety of organ types, including skin, liver, muscle, spleen, and pancreas. The gene gun transfer method is not dependent upon specific cell surface receptors, cell cycle status, or the size of the DNA vector. Useful gene gun devices include the Accelld (PowderJect Vaccines, Inc.) and the HelioSTM (Bio-Rad). These devices create a compressed shock wave of helium gas, accelerating DNA-coated gold (or tungsten) particles to high speed, whereby the particles have sufficient momentum to penetrate a target tissue.

Lipoplexes are typically made up of three components: a cationic lipid, a neutral colipid, and plasmid DNA that encodes one or more genes of interest. Commonly used cationic lipids include DOTMA, DMRIE, DC-chol, DOTAP, DMRIE, DDAB, DODAB/C, DOGS, DOSPA, SAINT-n, DOSPER, DPPES, DORIE, GAP-DLRIE, and DOTIM. Dioleoyl (DO) and dimyristoyl (DM) chains are thought to be especially effective for gene delivery. Cationic lipids are typically composed of a positively charged headgroup, a hydrophobic lipid anchor, and a linker that connects the headgroup and anchor. Catioinc lipids used in lipoplexes can be divided into two broad classes: those that use cholesterol as the lipid anchor and those that use diacyl chains of varying lengths and extent of saturation. The number of protonatable amines on the headgroup may affect transfection activity, with multivalent headgroups being generally more active than monovalent headgroups. The linker can be made of a variety of chemical structures, e.g., ether, amide, carbamate, amine, urea, ester, and peptide bonds. Neutral colipids of lipoplexes commonly include DOPE, DOPC, and cholesterol. Generally, DOPE is used as the neutral colipid with catioinc lipids that are based on cholesterol (e.g., DC-chol, GL-67) and cholesterol is used as the neutral colipid with cationic lipids that harbor-diacyl-chains as the hydrophobic anchor (e.g., DOTAP, DOTIM).

Polyplexes are formed when cationic polymers are mixed with DNA. Cationic polymers used to from polyplexes are of two general types: linear polymers such as polylysine and spermine; and the branched chain, spherical, or globular polycations such as polyethyleneimine and dendrimers. Lipopolyplexes are formed by the incorporation of polylysine into a lipoplex to form ternary complexes. DNA can be complexed with a natural biopolymer, e.g., gelatin or chitosan, functioning as a gene carrier to form nanospheres. Such biodegradable nanospheres have several advantages, including the coencapsulation of bioactive agents, e.g. nucleic acids and drugs, and the sustained release of the DNA. Gelatin-DNA or chitosan-DNA nanospheres are synthesized by mixing the DNA solution with an aqueous solution of gelatin or chitosan.

The effectiveness nonviral vectors may be enhanced by conjugation to ligands that direct the vector either to a particular cell type or to a particular location within a cell. Antibodies and other site-specific proteins can be attached to a vector, e.g., on the surface of the vector or incorporated in the membrane. Following injection, these vectors bind efficiently and specifically to a target site. With respect to liposomes, ligands to a cell surface receptor can be incorporated into the surface of a liposome by covalently modifying the ligand with a lipid group and adding it during the formation of liposomes. The following classes of ligands can be incorporated into the nonviral DNA delivery complexes of the invention in order to make them more effective for gene delivery: (1) peptides, e.g., peptides having a specific cell surface receptor so that complexes will be targeted to specific cells bearing the receptor; (2) nuclear localization signals, e.g., to promote efficient entry of DNA into the nucleus; (3) pH-sensitive ligands, to encourage endosomal escape; (4) steric stabilizing agents, to prevent destabilization of the complexes after introduction into the biological milieu. Gene chemistry approaches, e.g. peptide nucleic acids, can be used to couple ligands to DNA to improve the in vivo bioavailability and expression of the DNA.

In plasmid-based, non-viral gene delivery systems it is often useful to link a polypeptide (e.g., an antibody), nucleic acid molecule, or other compound to the gene delivery plasmid such that the polypeptide, nucleic acid molecule or other compound remains associated with the plasmid following intracellular delivery in a manner that does not interfere with the transcriptional activity of the plasmid. This can be accomplished using an appropriate biotin-conjugated peptide nucleic acid (PNA) clamp. A sequence complementary to the biotin-conjugated PNA clamp is inserted into the gene delivery plasmid. The biotin-conjugated PNA will bind essentially irreversibly to the complementary sequence inserted into the plasmid. A polypeptide, nucleic acid molecule or other compound of interest can be conjugated to streptavidin. The streptavidin conjugate can bind to the biotin-PNA clamp bound to the plasmid. In this manner, a polypeptide, nucleic acid molecule or other compound can be bound to a gene delivery plasmid such that the polypeptide, nucleic acid molecule or other compound remains bound to the plasmid even within a cell. Importantly, the PNA clamp-binding site in the plasmid must be chosen so as not to interfere with a needed promoter/enhancer or coding region or otherwise disrupt the expression of the gene in the plasmid. An alternative approach employs a maleimide-conjugated PNA clamp. Polypeptides, nucleic acid molecules and other compounds containing a free thiol residue may be conjugated directly to the maleimide-PNA-DNA hybrid. As with the biotin-conjugated method, this conjugation does not disturb the transcriptional activity of the plasmid if the PNA-binding site is chosen to be in a region of the plasmid not essential for gene activity. Both of these approaches are described in detail by Zelphati et al. ((2000) BioTechniques 28:304-315).

The host cells of the invention can also be used to produce nonhuman transgenic animals. For example, in one embodiment, a host cell of the invention is a fertilized oocyte or an embryonic stem cell into which PYRIN-9-coding sequences have been introduced. Such host cells can then be used to create non-human transgenic animals in which exogenous PYRIN-9 sequences have been introduced into their genome or homologous recombinant animals in which endogenous PYRIN-9 sequences have been altered. Such animals are useful for studying the function and/or activity of PYRIN-9 and for identifying and/or evaluating modulators of PYRIN-9 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous PYRIN-9 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing PYRIN-9-encoding nucleic acid into the male pronuclei of a fertilized oocyte, e.g., by microinjection, retroviral infection, and allowing the oocyte to develop in a pseudopregnant female foster animal. The PYRIN-9 cDNA sequence, e.g., that of SEQ ID NO:1 can be introduced as a transgene into the genome of a non-human animal. Alternatively, a nonhuman homolog or ortholog of the human PYRIN-9 gene, such as a mouse PYRIN-9 gene, can be isolated based on hybridization to the human PYRIN-9 cDNA and used as a transgene. Intronic sequences and polyadenylation signals can also be included in the transgene to increase the efficiency of expression of the transgene. A tissue-specific regulatory sequence(s) can be operably linked to the PYRIN-9 transgene to direct expression of PYRIN-9 protein to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the PYRIN-9 transgene in its genome and/or expression of PYRIN-9 mRNA in tissues or cells of the animals. A transgenic founder animal can then be used to breed additional animals carrying the transgene. Moreover, transgenic animals carrying a transgene encoding PYRIN-9 can further be bred to other transgenic animals carrying other transgenes.

To create an homologous recombinant animal, a vector is prepared which contains at least a portion of a PYRIN-9 gene (e.g., a human or a non-human homolog of the PYRIN-9 gene, e.g., a murine PYRIN-9 gene) into which a deletion, addition or substitution has been introduced to thereby alter, e.g., functionally disrupt, the PYRIN-9 gene. In an embodiment, the vector is designed such that, upon homologous recombination, the endogenous PYRIN-9 gene is functionally disrupted (i.e., no longer encodes a functional protein; also referred to as a “knock out” vector). Alternatively, the vector can be designed such that, upon homologous recombination, the endogenous PYRIN-9 gene is mutated or otherwise altered but still encodes functional protein (e.g., the upstream regulatory region can be altered to thereby alter the expression of the endogenous PYRIN-9 protein). In the homologous recombination vector, the altered portion of the PYRIN-9 gene is flanked at its 5′ and 3′ ends by additional nucleic acid of the PYRIN-9 gene to allow for homologous recombination to occur between the exogenous PYRIN-9 gene carried by the vector and an endogenous PYRIN-9 gene in an embryonic stem cell. The additional flanking PYRIN-9 nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced PYRIN-9 gene has homologously recombined with the endogenous PYRIN-9 gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

In another embodiment, the expression characteristics of an endogenous PYRIN-9 gene within a cell line or microorganism may be modified by inserting a heterologous DNA regulatory element into the genome of a stable cell line or cloned microorganism such that the inserted regulatory element is operatively linked with the endogenous PYRIN-9 gene. For example, an endogenous PYRIN-9 which is normally “transcriptionally silent,” i.e. a PYRIN-9 gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous PYRIN-9 gene may be activated by insertion of a promiscuous regulatory element that works across cell types.

A heterologous regulatory element may be inserted into a stable cell line or cloned microorganism, such that it is operatively linked with an endogenous PYRIN-9 gene, using techniques, such as targeted homologous recombination, which are well known to those of skill in the art, and described e.g., in Chappel, U.S. Pat. No. 5,272,071; PCT publication No. WO 91/06667, published May 16, 1991.

Pharmaceutical Compositions

The PYRIN-9 nucleic acid molecules, PYRIN-9 proteins, and anti-PYRIN-9 antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity may, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight les than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule-per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a PYRIN-9 protein or anti-PYRIN-9 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of protein, polypeptide, or antibody can include a single treatment or, preferably, can include a series of treatments.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The gene therapy vectors of the invention can be either viral or non-viral. Examples of plasmid-based, non-viral vectors are discussed in Huang et al. (1999) Nonviral Vectors for Gene Therapy (supra). A modified plasmid is one example of a non-viral gene delivery system. Peptides, proteins (including antibodies), and oligonucleotides may be stably conjugated to plasmid DNA by methods that do not interfere with the transcriptional activity of the plasmid (Zelphati et al. (2000) BioTechniques 28:304-315). The attachment of proteins and/or oligonucleotides may influence the delivery and trafficking of the plasmid and thus render it a more effective pharmaceutical composition.

The pharmaceutical compositions can be included in a container, pack, or dispenser together with instructions for administration.

Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology), c) predictive medicine (e.g., diagnostic assays, prognostic assays, monitoring clinical trials, and pharmacogenomics); and d) methods of treatment (e.g., therapeutic and prophylactic). A PYRIN-9 protein interacts with other cellular proteins and can thus be used for (i) regulation of cellular proliferation; (ii) regulation of cellular differentiation; and (iii) regulation of cell survival. The isolated nucleic acid molecules of the invention can be used to express PYRIN-9 protein (e.g., via a recombinant expression vector in a host cell in gene therapy applications), to detect PYRIN-9 mRNA (e.g., in a biological sample) or a genetic lesion in a PYRIN-9 gene, and to modulate PYRIN-9 activity. In addition, the PYRIN-9 proteins can be used to screen drugs or compounds which modulate the PYRIN-9 activity or expression as well as to treat disorders characterized by insufficient or excessive production of PYRIN-9 protein or production of PYRIN-9 protein forms which have decreased or aberrant activity compared to PYRIN-9 wild type protein. In addition, the anti-PYRIN-9 antibodies of the invention can be used to detect and isolate PYRIN-9 proteins and modulate PYRIN-9 activity.

This invention further pertains to novel agents identified by the above-described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) which bind to PYRIN-9 proteins or biologically active portions thereof or have a stimulatory or inhibitory effect on, for example, PYRIN-9 expression or PYRIN-9 activity. Examples of biologically active portions of human PYRIN-9 are domains described herein, such as a pyrin domain, an NBS domain (or a motif of an NBS domain), and a LRR domain (or a leucine rich repeat of a LRR domain).

Among the screening assays provided by the invention are screening to identify molecules that prevent the interaction of PYRIN-9 with another protein or biological molecule and screening to identify a competitive inhibitor of the binding of a nucleotide to the nucleotide binding site of PYRIN-9. Such assays can employ full-length PYRIN-9 or a portion of PYRIN-9, e.g., a domain or subdomain defined herein.

Molecules that bind to and/or alter the activity of an NBS domain of PYRIN-9 may be useful for modulating the activity of PYRIN-9. For example, molecules can be tested for their ability to modulate, e.g., antagonize, the hydrolysis of a nucleotide triphosphate (NTP), e.g., ATP or GTP, by the NBS domain (or a fragment of an NBS domain such as an NBS motif described herein) of PYRIN-9. Methods of detecting the hydrolysis of a NTP by a protein containing a nucleotide-binding site are described in, for example, Li et al. (1996) J. Biol. Chem. 271:28463-28468 and Gadsby et al. (1999) Physiol. Rev. 79:S77-S107.

A purified protein containing an NBS domain of PYRIN-9 can be evaluated for its ability to mediate NTPase activity in vitro. The assay can be performed in the presence of a test compound to determine the ability of the test compound to modulate the NTPase activity of the purified protein. In addition, or alternatively, the purified protein used in an NTPase activity assay can be a variant or a fragment of PYRIN-9, and the assay can be performed to determine the NTPase activity of the fragment or variant.

In one example, an NBS domain can be assayed for its ability to hydrolyze ATP. ATPase activity can measured as the production of [I³²-P]ADP from [I³²-P]ATP, using polyethyleneimine-cellulose chromatography for separation of the nucleotides. The assay can be carried out in a 15 Ti reaction mixture containing 50 mM Tris, 50 mM NaCl, pH 7.5, 2 mM MgCl₂, 10% glycerol, 0.5 mM CHAPS, and 8 TCi of [I³²-P]ATP. Reaction mixtures are incubated at 30° C. and are stopped by the addition of 5 Tl of 10% SDS. One Tl samples are spotted on a polyethyleneimine-cellulose plate and developed in 1 M formic acid, 0.5 M LiCl. The location and quantitation of the radiolabeled ATP and ADP can determined with a STORM PHOSPHORIMAGER gel and blot imaging system (Amersham Biosciences, Piscataway, N.J.). Data can be analyzed using the IMAGEQUANT image analysis software package (Amersham). See, e.g., Li et al. (1996) J. Biol. Chem. 271:28463-28468 for additional details on methods detecting ATPase activity by nucleotide binding site-containing proteins and variants thereof. Thin layer chromatography techniques similar to those described above can also be used for the measurement of NTPase activity such as GTPase activity (see, e.g., Gout et al. (1993) Cell 75:25-36).

Screening assays can be used to identify molecules that bind to and/or modulate the activity of a pyrin domain or a LRR domain of a PYRIN-9 protein, fragment, or variant thereof.

Screening assays can be used to identify molecules that modulate the phosphorylation state of a PYRIN-9 protein, fragment, or variant thereof. Accordingly, the phosphorylation or dephosphorylation of the PYRIN-9 protein, fragment, or variant thereof in the presence of the test compound can be detected in a suitable assay and compared to the result in the absence of the test compound.

Screening assays can also be used to identify molecules which modulate a PYRIN-9 mediated increase in transcription of genes having an AP-1 or NF-κB binding site. For example, expression of a reporter gene under the control of NF-κB (or AP-1) is measured in the presence and absence of a candidate molecule and in the presence and absence of PYRIN-9 to identify those molecules which alter expression of the reporter in a PYRIN-9 dependent manner. In addition, screening assays can be used to identify molecules that modulate a PYRIN-9 mediated increase in CHOP phosphorylation. For example, the expression of a reporter gene under the control of CHOP is measured in the presence and absence of a candidate small molecule and in the presence and absence of PYRIN-9 to identify those molecules that alter expression of the reporter in a PYRIN-9 dependent manner. A screening assay can be carried out to identify molecules which modulate the PYRIN-9 mediated increase in CHOP phosphorylation. For example, CHOP phosphorylation is measured in the presence and absence of a candidate molecule and in the presence and absence of PYRIN-9. Phosphorylation of CHOP can be measured using an antibody which binds to phosphorylated CHOP, but not to non-phosphorylated CHOP.

In one embodiment, the invention provides assays for screening candidate or test compounds-which bind to or modulate the activity of a PYRIN-9 proteins or polypeptides or biologically active portions thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is one in which a polypeptide of the invention, or a biologically active portion thereof, is contacted with a test compound and the ability of the test compound to bind to the polypeptide determined. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

Determining the ability of the test compound to modulate the activity of PYRIN-9 or a biologically active portion thereof can be accomplished, for example, by determining the ability of the PYRIN-9 protein to bind to or interact with a PYRIN-9 target molecule. As used herein, a “target molecule” is a molecule with which a PYRIN-9 protein binds or interacts in nature, for example, a molecule associated with the internal surface of a cell membrane or a cytoplasmic molecule. A PYRIN-9 target molecule can be a non-PYRIN-9 molecule or a PYRIN-9 protein or polypeptide of the present invention. In one embodiment, a PYRIN-9 target molecule is a component of an apoptotic signal transduction pathway. The target, for example, can be a second intracellular protein which has catalytic activity or a protein which facilitates the association of downstream signaling molecules with PYRIN-9. In particular the target can be another protein having a pyrin domain (or a pyrin domain containing fragment thereof (e.g. CARD-5 or a polypeptide comprising the pyrin domain of CARD-5)).

Determining the ability of the test compound to modulate the activity of PYRIN-9 or a biologically active portion thereof can be accomplished, for example, by determining the ability of the PYRIN-9 protein to bind to or interact with any of the specific proteins listed in the previous paragraph as PYRIN-9 target molecules. In another embodiment, PYRIN-9 target molecules include all proteins that bind to a PYRIN-9 protein or a fragment thereof in a two-hybrid system binding assay which can be used without undue experimentation to isolate such proteins from cDNA or genomic two-hybrid system libraries. The binding assays described in this section can be cell-based or cell free (described subsequently).

Determining the ability of the PYRIN-9 protein to bind to or interact with a PYRIN-9 target molecule can be accomplished by one of the methods described above for determining direct binding. In an embodiment, determining the ability of the PYRIN-9 protein to bind to or interact with a PYRIN-9 target molecule can be accomplished by determining the activity of the target molecule. For example, the activity of the target molecule can be determined by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a PYRIN-9-responsive regulatory element operatively linked to a nucleic acid encoding a detectable marker, e.g. luciferase), or detecting a cellular response, for example, cell survival, cellular differentiation, or cell proliferation. The activity of a target molecule can be monitored by assaying the apoptosis cellular response (e.g. mediated by caspase 9) or assaying enzymatic activity, e.g. caspase 1 enzymatic activity, e.g. cytokine processing (e.g. cleavage of pro-IL-1β or secretion of bioactive (IL-1β). In addition, and in another embodiment, genes induced by PYRIN-9 expression can be identified by expressing PYRIN-9 in a cell line and conducting a transcriptional profiling experiment wherein the mRNA expression patterns of the cell line transformed with an empty expression vector and the cell line transformed with a PYRIN-9 expression vector are compared. The promoters of genes induced by PYRIN-9 expression can be operatively linked to reporter genes suitable for screening such as luciferase, secreted alkaline phosphatase, or beta-galactosidase and the resulting constructs could be introduced into appropriate expression vectors. A recombinant cell line containing PYRIN-9 and transfected with an expression vector containing a PYRIN-9 responsive promoter operatively linked to a reporter gene can be used to identify test compounds that modulate PYRIN-9 activity by assaying the expression of the reporter gene in response to contacting the recombinant cell line with test compounds. PYRIN-9 agonists can be identified as increasing the expression of the reporter gene and PYRIN-9 antagonists can be identified as decreasing the expression of the reporter gene.

In another embodiment of the invention, the ability of a test compound to modulate the activity of PYRIN-9, or biologically active portions thereof can be determined by assaying the ability of the test compound to modulate PYRIN-9-dependent pathways or processes where the PYRIN-9 target proteins that mediate the PYRIN-9 effect are known or unknown. Potential PYRIN-9-dependent pathways or processes include, but are not limited to, the modulation of cellular signal transduction pathways and their related second messenger molecules (e.g., intracellular Ca²⁺, diacylglycerol, IP3, cAMP etc.), cellular enzymatic activities, cellular responses (e.g., cell survival, cellular differentiation, or cell proliferation), or the induction or repression of cellular or heterologous mRNAs or proteins. PYRIN-9-dependent pathways or processes could be assayed by standard cell-based or cell free assays appropriate for the specific pathway or process under study. In another embodiment, cells cotransfected with PYRIN-9, CARD-5 and a NF-κB luciferase reporter gene could be contacted with a test compound and test compounds that block PYRIN-9 activity could be identified by their reduction of PYRIN-9-dependent NF-κB pathway luciferase reporter gene expression. Test compounds that agonize PYRIN-9 would be expected to increase reporter gene expression. In another embodiment, PYRIN-9 could be expressed in a cell line and the recombinant PYRIN-9-expressing cell line could be contacted with a test compound. Test compounds that inhibit PYRIN-9 activity could be identified by their reduction of PYRIN-9-depended NF-κB pathway stimulation as measured by the assay of a NF-κB pathway reporter gene, NF-κB nuclear localization, IκB phosphorylation or proteolysis, or other standard assays for NF-κB pathway activation known to those skilled in the art.

In yet another embodiment, an assay of the present invention is a cell-free assay comprising contacting a PYRIN-9 protein or biologically active portion thereof with a test compound and determining the ability of the test compound to bind to the PYRIN-9 protein or biologically active portion thereof. Binding of the test compound to the PYRIN-9 protein can be determined either directly or indirectly as described above. In one embodiment, a competitive binding assay includes contacting the PYRIN-9 protein or biologically active portion thereof with a compound known to bind PYRIN-9 to form an assay mixture, contacting the assay mixture with a test compound, and determining the ability of the test compound to interact with a PYRIN-9 protein, wherein determining the ability of the test compound to interact with a PYRIN-9 protein comprises determining the ability of the test compound to preferentially bind to PYRIN-9 or biologically active portion thereof as compared to the known binding compound.

In another embodiment, an assay is a cell-free assay comprising contacting PYRIN-9 protein or biologically active portion thereof with a test compound and determining the ability of the test compound to modulate (e.g., stimulate or inhibit) the activity of the PYRIN-9 protein or biologically active portion thereof. Determining the ability of the test compound to modulate the activity of PYRIN-9 can be accomplished, for example, by determining the ability of the PYRIN-9 protein to bind to or interact with a PYRIN-9 target molecule by one of the methods described above for determining direct binding. In an alternative embodiment, determining the ability of the test compound to modulate the activity of PYRIN-9 can be accomplished by determining the ability of the PYRIN-9 protein to further modulate a PYRIN-9 target molecule. For example, the catalytic/enzymatic activity of the target molecule on an appropriate substrate can be determined as previously described.

In yet another embodiment, the cell-free assay comprises contacting the PYRIN-9 protein or biologically active portion thereof with a known compound which binds PYRIN-9 to form an assay mixture, contacting the assay-mixture with a test compound, and determining the ability of the test compound to interact with a PYRIN-9 protein, wherein determining the ability of the test compound to interact with a PYRIN-9 protein comprises determining the ability of the PYRIN-9 protein to preferentially bind to or modulate the activity of a PYRIN-9 target molecule. The cell-free assays of the present invention are amenable to use of either the soluble form or a membrane-associated form of PYRIN-9. A membrane-associated form of PYRIN-9 refers to PYRIN-9 that interacts with a membrane-bound target molecule. In the case of cell-free assays comprising the membrane-associated form of PYRIN-9, it may be desirable to utilize a solubilizing agent such that the membrane-associated form of PYRIN-9 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either PYRIN-9 or its target molecule to facilitate separation of complexed from uncomplexed forms of one or both of the proteins, as well as to accommodate automation of the assay. Binding of a test compound to PYRIN-9, or interaction of PYRIN-9 with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/PYRIN-9 fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or PYRIN-9 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of PYRIN-9 binding or activity determined using standard techniques. In an alternative embodiment, MYC or HA epitope tag PYRIN-9 fusion proteins or MYC or HA epitope tag target fusion proteins can be adsorbed onto anti-MYC or anti-HA antibody coated microbeads or onto anti-MYC or anti-HA antibody coated microtitre plates, which are then combined with the test compound or the test compound and either the non-adsorbed target protein or PYRIN-9 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of PYRIN-9 binding or activity determined using standard techniques.

Other techniques for immobilizing proteins on matrices can also be used in the screening assays of the invention. For example, PYRIN-9 or its target molecule can be immobilized utilizing conjugation of biotin and streptavidin. Biotinylated PYRIN-9 target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with PYRIN-9 or target molecules but which do not interfere with binding of the protein to its target molecule can be derivatized to the wells of the plate, and unbound target or protein trapped in the wells by antibody conjugation. Methods for detecting such complexes, in addition to those described above for the GST-immobilized complexes and epitope tag immobilized complexes, include immunodetection of complexes using antibodies reactive with the PYRIN-9 or target molecule, as well as enzyme-linked assays which rely on detecting an enzymatic activity associated with the PYRIN-9 or a target molecule.

In another embodiment, modulators of PYRIN-9 expression are identified in a method in which a cell is contacted with a candidate compound and the expression of the PYRIN-9 promoter, mRNA or protein in the cell is determined. The level of expression of PYRIN-9 mRNA or protein in the presence of the candidate compound is compared to the level of expression of PYRIN-9 mRNA or protein in the absence of the candidate compound. The candidate compound can then be identified as a modulator of PYRIN-9 expression based on this comparison. For example, when expression of PYRIN-9 mRNA or protein is greater (statistically significantly greater) in the presence of the candidate compound than in its absence, the candidate compound is identified as a stimulator of PYRIN-9 mRNA or protein expression. Alternatively, when expression of PYRIN-9 mRNA or protein is less (statistically significantly less) in the presence of the candidate compound than in its absence, the candidate compound is identified as an inhibitor of PYRIN-9 mRNA or protein expression. The level of PYRIN-9 mRNA or protein expression in the cells can be determined by methods described herein for detecting PYRIN-9 mRNA or protein. The activity of the PYRIN-9 promoter can be assayed by linking the PYRIN-9 promoter to a reporter gene such as luciferase, secreted alkaline phosphatase, or beta-galactosidase and introducing the resulting construct into an appropriate vector, transfecting a host cell line, and measuring the activity of the reporter gene in response to test compounds.

In yet another aspect of the invention, the PYRIN-9 proteins can be used as “bait proteins” in a two-hybrid assay (for a discussion of a mammalian two-hybrid assay, see e.g., Hosfield and Chang (1999) Strategies Newsletter 2(2):62-65) or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with PYRIN-9 (“PYRIN-9-binding proteins” or “PYRIN-9-bp”) and modulate PYRIN-9 activity. Such PYRIN-9-binding proteins are also likely to be involved in the propagation of signals by the PYRIN-9 proteins as, for example, upstream or downstream elements of the PYRIN-9 pathway.

The two-hybrid system is based on the modular nature of most transcription factors, which consist of separable DNA-binding and activation domains. Briefly, the assay utilizes two different DNA constructs. In one construct, the gene that codes for PYRIN-9 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PYRIN-9-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with PYRIN-9.

In an embodiment of the invention, the ability of a test compound to modulate the activity of PYRIN-9, or a biologically active portion thereof can be determined by assaying the ability of the test compound to block the binding of PYRIN-9 to its target proteins in a yeast or mammalian two-hybrid system assay. This assay could be automated for high throughput drug screening purposes. In another embodiment of the invention, PYRIN-9 and a target protein could be configured in the reverse two-hybrid system (Vidal et al. (1996) Proc. Natl. Acad. Sci. USA 93:10321-6 and Vidal et al. (1996) Proc. Natl. Acad. Sci. USA 93:10315-20) designed specifically for efficient drug screening. In the reverse two-hybrid system, inhibition of a PYRIN-9 physical interaction with a target protein would result in induction of a reporter gene in contrast to the normal two-hybrid system where inhibition of PYRIN-9 physical interaction with a target protein would lead to reporter gene repression. The reverse two-hybrid system is preferred for drug screening because reporter gene induction is more easily assayed than report gene repression.

Alternative embodiments of the invention are proteins found to physically interact with proteins that bind to PYRIN-9. PYRIN-9 interactors could be configured into two-hybrid system baits and used in two-hybrid screens to identify additional members of the PYRIN-9 pathway. The interactors of PYRIN-9 interactors identified in this way could be useful targets for therapeutic intervention in PYRIN-9 related diseases and pathologies and an assay of their enzymatic or binding activity could be useful for the identification of test compounds that modulate PYRIN-9 activity.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

Chromosome Mapping

Once the sequence (or a portion of the sequence) of a gene has been isolated, this sequence can be used to map the location of the gene on a chromosome. Accordingly, PYRIN-9 nucleic acid molecules described herein or fragments thereof, can be used to map the location of PYRIN-9 genes on a chromosome. The mapping of the PYRIN-9 sequences to chromosomes is an important first step in correlating these sequences with genes associated with disease.

Briefly, PYRIN-9 genes can be mapped to chromosomes by preparing PCR primers (preferably 15-25 bp in length) from the PYRIN-9 sequences. Computer analysis of PYRIN-9 sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the PYRIN-9 sequences will yield an amplified fragment. Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced using human chromosomes with translocations and deletions.

PCR mapping of somatic cell hybrids is a rapid procedure for assigning a particular sequence to a particular chromosome. Three or more sequences can be assigned per day using a single thermal cycler. Using the PYRIN-9 sequences to design oligonucleotide primers, sublocalization can be achieved with panels of fragments from specific chromosomes. Other mapping strategies which can similarly be used to map a PYRIN-9 sequence to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature, 325:783-787.

Moreover, differences in the DNA sequences between individuals affected and unaffected with a disease associated with the PYRIN-9 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

A PYRIN-9 polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of the PYRIN-9 polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

Tissue Typing

The PYRIN-9 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the PYRIN-9 sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The PYRIN-9 sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of PYRIN-9 can comfortably provide positive individual identification with a panel of perhaps 10 to 1,000 primers which each yield a noncoding amplified sequence of 100 bases. If predicted coding sequences, such as those in SEQ ID NO:1 are used, a more appropriate number of primers for positive individual identification would be 500-2,000.

If a panel of reagents from PYRIN-9 sequences described herein is used to generate a unique identification database for an individual, those same reagents can later be used to identify tissue from that individual. Using the unique identification database, positive identification of the individual, living or dead, can be made from extremely small tissue samples.

Use of Partial Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of PYRIN-9 are particularly appropriate for this use as greater numbers of polymorphisms occur in the noncoding regions, making it easier to differentiate individuals using this technique. Examples of polynucleotide reagents include the PYRIN-9 sequences or portions thereof, e.g., fragments derived from the noncoding regions of PYRIN-9 which have a length of at least 20 or 30 bases.

The sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such PYRIN-9 probes can be used to identify tissue by species and/or by organ type.

In a similar fashion, these reagents, e.g., PYRIN-9 primers or probes can be used to screen tissue culture for contamination (i.e., screen for the presence of a mixture of different types of cells in a culture).

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining PYRIN-9 protein and/or nucleic acid expression as well as PYRIN-9 activity, in the context of a biological sample (e.g., blood, serum, cells, tissue) to thereby determine whether an individual is afflicted with a disease or disorder, or is at risk of developing a disorder, associated with aberrant PYRIN-9 expression or activity. The invention also provides for prognostic (or predictive) assays for determining whether an individual is at risk of developing a disorder associated with PYRIN-9 protein, nucleic acid expression or activity. For example, mutations in a PYRIN-9 gene can be assayed in a biological sample. Such assays can be used for prognostic or predictive purpose to thereby prophylactically treat an individual prior to the onset of a disorder characterized by or associated with PYRIN-9 protein, nucleic acid expression or activity.

Another aspect of the invention provides methods for determining PYRIN-9 protein, nucleic acid expression or PYRIN-9 activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of PYRIN-9 in clinical trials.

These and other agents are described in further detail in the following sections.

Diagnostic Assays

An exemplary method for detecting the presence or absence of PYRIN-9 in a biological sample involves obtaining a biological sample from a test subject and contacting the biological sample with a compound or an agent capable of detecting PYRIN-9 protein or nucleic acid (e.g., mRNA, genomic DNA) that encodes PYRIN-9 protein such that the presence of PYRIN-9 is detected in the biological sample. An agent for detecting PYRIN-9 mRNA or genomic DNA is a labeled nucleic acid probe capable of hybridizing to PYRIN-9 mRNA or genomic DNA. The nucleic acid probe can be, for example, the nucleic acid of SEQ ID NO:1 or a portion thereof, such as an oligonucleotide of at least 15, 30, 50, 100, 250, 500, 750, 1000, 1250, or 1500 nucleotides in length and sufficient to specifically hybridize under stringent conditions to mRNA or genomic DNA. Other suitable probes for use in the diagnostic assays of the invention are described herein.

An agent for detecting PYRIN-9 protein can be an antibody capable of binding to PYRIN-9 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells, biological fluids, and stool samples isolated from a subject, as well as tissues, cells and fluids present within a subject. That is, the detection method of the invention can be used to detect PYRIN-9 mRNA, protein, or genomic DNA in a biological sample in vitro as well as in vivo. For example, in vitro techniques for detection of PYRIN-9 mRNA include Northern hybridizations and in situ hybridizations. In vitro techniques for detection of PYRIN-9 protein include enzyme linked immunosorbent assays (ELISAs), Western blots, immunoprecipitations and immunofluorescence. In vitro techniques for detection of PYRIN-9 genomic DNA include Southern hybridizations. Furthermore, in vivo techniques for detection of PYRIN-9 protein include introducing into a subject a labeled anti-PYRIN-9 antibody. For example, the antibody can be labeled with a radioactive marker whose presence and location in a subject can be detected by standard imaging techniques.

Stool samples may be analyzed using various in vitro techniques, including techniques directed to analysis of DNA, RNA, or protein in the sample (Machiels et al. (2000) BioTechniques 28:286-290).

In one embodiment, the biological sample contains protein molecules from the test subject. Alternatively, the biological sample can contain mRNA molecules from the test subject or genomic DNA molecules from the test subject. A biological sample is a peripheral blood leukocyte sample isolated by conventional means from a subject.

In another embodiment, the methods further involve obtaining a control biological sample from a control subject, contacting the control sample with a compound or agent capable of detecting PYRIN-9 protein, mRNA, or genomic DNA, such that the presence of PYRIN-9 protein, mRNA or genomic DNA is detected in the biological sample, and comparing the presence of PYRIN-9 protein, mRNA or genomic DNA in the control sample with the presence of PYRIN-9 protein, mRNA or genomic DNA in the test sample.

The invention also encompasses kits for detecting the presence of PYRIN-9 in a biological sample (a test sample). Such kits can be used to determine if a subject is suffering from or is at increased risk of developing a disorder associated with aberrant expression of PYRIN-9 (e.g., an immunological disorder). For example, the kit can comprise a labeled compound or agent capable of detecting PYRIN-9 protein or mRNA in a biological sample and means for determining the amount of PYRIN-9 in the sample (e.g., an anti-PYRIN-9 antibody or an oligonucleotide probe which binds to DNA encoding PYRIN-9, e.g., SEQ ID NO:1). Kits may also include instruction for observing that the tested subject is suffering from or is at risk of developing a disorder associated with aberrant expression of PYRIN-9 if the amount of PYRIN-9 protein or mRNA is above or below a normal level.

For antibody-based kits, the kit may comprise, for example: (1) a first antibody (e.g., attached to a solid support) which binds to PYRIN-9 protein; and, optionally, (2) a second, different antibody which binds to PYRIN-9 protein or the first antibody and is conjugated to a detectable agent. For oligonucleotide-based kits, the kit may comprise, for example: (1) a oligonucleotide, e.g., a detectably labeled oligonucleotide, which hybridizes to a PYRIN-9 nucleic acid sequence or (2) a pair of primers useful for amplifying a PYRIN-9 nucleic acid molecule.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of PYRIN-9.

Prognostic Assays

The methods described herein can furthermore be utilized as diagnostic or prognostic assays to identify subjects having or at risk of developing a disease or disorder associated with aberrant PYRIN-9 expression or activity. For example, the assays described herein, such as the preceding diagnostic assays or the following assays, can be utilized to identify a subject having or at risk of developing a disorder associated with PYRIN-9 protein, nucleic acid expression or activity. Alternatively, the prognostic assays can be utilized to identify a subject having or at risk for developing such a disease or disorder. Thus, the present invention provides a method in which a test sample is obtained from a subject and PYRIN-9 protein or nucleic acid (e.g., mRNA, genomic DNA) is detected, wherein the presence of PYRIN-9 protein or nucleic acid is diagnostic for a subject having or at risk of developing a disease or disorder associated with aberrant PYRIN-9 expression or activity. As used herein, a “test sample” refers to a biological sample obtained from a subject of interest. For example, a test sample can be a biological fluid (e.g., serum), cell sample, tissue, or stool sample. Stool samples may be analyzed using various in vitro techniques, including techniques directed to analysis of DNA, RNA, or protein in the sample (Machiels et al. (2000) BioTechniques 28:286-290). Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant PYRIN-9 expression or activity. For example, such methods can be used to determine whether a subject can be effectively treated with a specific agent or class of agents (e.g., agents of a type which decrease PYRIN-9 activity). Thus, the present invention provides methods for determining whether a subject can be effectively treated with an agent for a disorder associated with aberrant PYRIN-9 expression or activity in which a test sample is obtained and PYRIN-9 protein or nucleic acid is detected (e.g., wherein the presence of PYRIN-9 protein or nucleic acid is diagnostic for a subject that can be administered the agent to treat a disorder associated with aberrant PYRIN-9 expression or activity).

The methods of the invention can also be used to detect genetic lesions or mutations in a PYRIN-9 gene, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion characterized by at least one of an alteration affecting the integrity of a gene encoding a PYRIN-9-protein, or the mis-expression of the PYRIN-9 gene. For example, such genetic lesions can be detected by ascertaining the existence of at least one of 1) a deletion of one or more nucleotides from a PYRIN-9 gene; 2) an addition of one or more nucleotides to a PYRIN-9 gene; 3) a substitution of one or more nucleotides of a PYRIN-9 gene; 4) a chromosomal rearrangement of a PYRIN-9 gene; 5) an alteration in the level of a messenger RNA transcript of a PYRIN-9 gene; 6) aberrant modification of a PYRIN-9 gene, such as of the methylation pattern of the genomic DNA; 7) the presence of a non-wild type splicing pattern of a messenger RNA transcript of a PYRIN-9 gene (e.g., caused by a mutation in a splice donor or splice acceptor site); 8) a non-wild type level of a PYRIN-9-protein; 9) allelic loss of a PYRIN-9 gene; and 10) inappropriate post-translational modification of a PYRIN-9-protein. As described herein, there are a large number of assay techniques known in the art which can be used for detecting lesions in a PYRIN-9 gene. A biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the PYRIN-9 gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a PYRIN-9 gene under conditions such that hybridization and amplification of the PYRIN-9-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a PYRIN-9 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in PYRIN-9 can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in PYRIN-9 can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the PYRIN-9 gene and detect mutations by comparing the sequence of the sample PYRIN-9 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the PYRIN-9 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type PYRIN-9 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al (1988) Proc. Natl. Acad Sci USA 85:4397; Saleeba et al (1992) Methods Enzymol. 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in PYRIN-9 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a PYRIN-9 sequence, e.g., a wild-type PYRIN-9 sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in PYRIN-9 genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet Anal Tech Appl 9:73-79). Single-stranded DNA fragments of sample and control PYRIN-9 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In an embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting-point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a PYRIN-9 gene.

Furthermore, any cell type or tissue, preferably peripheral blood leukocytes, in which PYRIN-9 is expressed may be utilized in the prognostic assays described herein.

Pharmacogenomics

Agents, or modulators which have a stimulatory or inhibitory effect on PYRIN-9 activity (e.g., PYRIN-9 gene expression) as identified by a screening assay described herein can be administered to individuals to treat (prophylactically or therapeutically) disorders (e.g., an immune or inflammatory disorder or a neurodegenerative disease such as Alzheimer's disease) associated with aberrant PYRIN-9 activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of PYRIN-9 protein, expression of PYRIN-9 nucleic acid, or mutation content of PYRIN-9 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM exhibit no therapeutic response, as demonstrated for the analgesic effect of codeine mediated by its CYP2D6-formed metabolite morphine. The other extreme are the so-called ultra-rapid metabolizers who do not respond to standard doses. Recently, the molecular basis of ultra-rapid metabolism has been identified to be due to CYP2D6 gene amplification.

Thus, the activity of PYRIN-9 protein, expression of PYRIN-9 nucleic acid, or mutation content of PYRIN-9 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a PYRIN-9 modulator, such as a modulator identified by one of the exemplary screening assays described herein.

Monitoring of Effects During Clinical Trials

Monitoring the influence of agents (e.g., drugs, compounds) on the expression or activity of PYRIN-9 (e.g., the ability to modulate aberrant cell proliferation and/or differentiation) can be applied not only in basic drug screening, but also in clinical trials. For example, the effectiveness of an agent determined by a screening assay as described herein to increase PYRIN-9 gene expression, protein levels, or upregulate PYRIN-9 activity, can be monitored in clinical trails of subjects exhibiting decreased PYRIN-9 gene expression, protein levels, or downregulated PYRIN-9 activity. Alternatively, the effectiveness of an agent determined by a screening assay to decrease PYRIN-9 gene expression, protein levels, or downregulated PYRIN-9 activity, can be monitored in clinical trials of subjects exhibiting increased PYRIN-9 gene expression, protein levels, or upregulated PYRIN-9 activity. In such clinical trials, the expression or activity of PYRIN-9 and, preferably, other genes that have been implicated in, for example, a cellular proliferation disorder can be used as a “read out” or markers of the immune responsiveness of a particular cell.

For example, and not by way of limitation, genes, including PYRIN-9, that are modulated in cells by treatment with an agent (e.g., compound, drug or small molecule) which modulates PYRIN-9 activity (e.g., identified in a screening assay as described herein) can be identified. Thus, to study the effect of agents on cellular proliferation disorders, for example, in a clinical trial, cells can be isolated and RNA prepared and analyzed for the levels of expression of PYRIN-9 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of PYRIN-9 or other genes. In this way, the gene expression pattern can serve as a marker, indicative of the physiological response of the cells to the agent. Accordingly, this response state may be determined before, and at various points during, treatment of the individual with the agent.

In an embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a PYRIN-9 protein, mRNA, or genomic DNA in the preadministration sample; (iii) obtaining one or more post-administration samples from the subject; (iv) detecting the level of expression or activity of the PYRIN-9 protein, mRNA, or genomic DNA in the post-administration samples; (v) comparing the level of expression or activity of the PYRIN-9 protein, mRNA, or genomic DNA in the pre-administration sample with the PYRIN-9 protein, mRNA, or genomic DNA in the post administration sample or samples; and (vi) altering the administration of the agent to the subject accordingly. For example, increased administration of the agent may be desirable to increase the expression or activity of PYRIN-9 to higher levels than detected, i.e., to increase the effectiveness of the agent. Alternatively, decreased administration of the agent may be desirable to decrease expression or activity of PYRIN-9 to lower levels than detected, i.e., to decrease the effectiveness of the agent.

Transcriptional Profiling

The PYRIN-9 nucleic acid molecules described herein, including small oligonucleotides, can be used in transcriptionally profiling. For example, these nucleic acids can be used to examine the expression of PYRIN-9 in normal-tissue or cells and in tissue or cells subject to a disease state, e.g., tissue or cells derived from a patient having a disease of interest or cultured cells which model or reflect a disease state of interest, e.g., cells of a cultured tumor cell line. By measuring expression of PYRIN-9, together or individually, a profile of expression in normal and disease states can be developed. This profile can be used diagnostically and to examine the effectiveness of a therapeutic regime.

Methods of Treatment

The present invention provides for both prophylactic and therapeutic methods of treating a subject at risk of (or susceptible to) a disorder or having a disorder associated with aberrant PYRIN-9 expression or activity, examples of which are provided herein.

Prophylactic Methods

In one aspect, the invention provides a method for preventing in a subject, a disease or condition associated with an aberrant PYRIN-9 expression or activity, by administering to the subject an agent which modulates PYRIN-9 expression or at least one PYRIN-9 activity. Subjects at risk for a disease which is caused or contributed to by aberrant PYRIN-9 expression or activity can be identified by, for example, any or a combination of diagnostic or prognostic assays as described herein. Administration of a prophylactic agent can occur prior to the manifestation of symptoms characteristic of the PYRIN-9 aberrancy, such that a disease or disorder is prevented or, alternatively, delayed in its progression. Depending on the type of PYRIN-9 aberrancy, for example, a PYRIN-9 agonist or PYRIN-9 antagonist agent can be used for treating the subject. The appropriate agent can be determined based on screening assays described herein.

Therapeutic Methods

Another aspect of the invention pertains to methods of modulating PYRIN-9 expression or activity for therapeutic purposes. The modulatory method of the invention involves contacting a cell with an agent that modulates one or more of the activities of PYRIN-9 protein activity associated with the cell. An agent that modulates PYRIN-9 protein activity can be an agent as described herein, such as a nucleic acid or a protein, a naturally-occurring cognate ligand of a PYRIN-9 protein, a peptide, a PYRIN-9 peptidomimetic, or other small molecule. In one embodiment, the agent stimulates one or more of the biological activities of PYRIN-9 protein. Examples of such stimulatory agents include active PYRIN-9 protein and a nucleic acid molecule encoding PYRIN-9 that has been introduced into the cell. In another embodiment, the agent inhibits one or more of the biological activities of PYRIN-9 protein. Examples of such inhibitory agents include antisense PYRIN-9 nucleic acid molecules and anti-PYRIN-9 antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a PYRIN-9 protein or nucleic acid molecule or a disorder related to PYRIN-9 expression or activity. In one embodiment, the method involves administering an agent (e.g., an agent identified by a screening assay described herein), or combination of agents that modulates (e.g., upregulates or downregulates) PYRIN-9 expression or activity. In another embodiment, the method involves administering a PYRIN-9 protein or nucleic acid molecule as therapy to compensate for reduced or aberrant PYRIN-9 expression or activity. Stimulation of PYRIN-9 activity is desirable in situations in which PYRIN-9 is abnormally downregulated and/or in which increased PYRIN-9 activity is likely to have a beneficial effect. Conversely, inhibition of PYRIN-9 activity is desirable in situations in which PYRIN-9 is abnormally upregulated, e.g., in myocardial infarction, and/or in which decreased PYRIN-9 activity is likely to have a beneficial effect.

This invention is further illustrated by the following examples which should not be construed as limiting. The contents of all references, patents and published patent applications cited throughout this application are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence of SEQ ID NO:1;
b) a nucleic acid molecule which encodes a polypeptide comprising the amino acid sequence of SEQ ID NO:2;
c) a nucleic acid molecule which encodes a polypeptide fragment comprising at least 25 amino acids of the amino acid sequence of SEQ ID NO:2, wherein the fragment has at least one biological activity of PYRIN-9;
d) nucleic acid molecule comprising at least 75 contiguous nucleotides of SEQ ID NO:1, wherein the nucleic acid molecule encodes a polypeptide which has at least one biological activity of PYRIN-9; and
e) a nucleic acid molecule which encodes a naturally occurring allelic variant of a polypeptide comprising the amino acid sequence of SEQ ID NO:2, wherein the nucleic acid molecule hybridizes to a nucleic acid molecule comprising SEQ ID NO:1, or a complement thereof, under stringent conditions and the variant has at least one biological activity of PYRIN-9.

2. The nucleic acid molecule of claim 1 further comprising vector nucleic acid sequences.

3. The nucleic acid molecule of claim 2 wherein the vector nucleic acid sequences comprise expression control sequences.

4. A host cell which expresses the nucleic acid molecule of claim 1.

5. An isolated polypeptide selected from the group consisting of:
a) a polypeptide which is encoded by a nucleic acid molecule comprising a nucleotide sequence of SEQ ID NO:1, or a complement thereof;
b) a polypeptide comprising the amino acid sequence of SEQ ID NO:2;
c) a naturally occurring allelic variant of a polypeptide comprising the amino acid sequence of SEQ ID NO:2, wherein the polypeptide is encoded by a nucleic acid molecule which hybridizes to a nucleic acid molecule comprising SEQ ID NO:1, or a complement thereof under stringent conditions and the variant has at least one biological activity of PYRIN-9;
d) a polypeptide comprising the amino acid sequence of SEQ ID NO:2 wherein up to 10 amino acids are replaced by conservative substitutions; and
e) a fragment of a polypeptide comprising the amino acid sequence of SEQ ID NO:2, wherein the fragment comprises at least 25 contiguous amino acids of SEQ ID NO:2, and the fragment has at least one biological activity of PYRIN-9.

6. The polypeptide of claim 5, further comprising heterologous amino acid sequences.

7. An antibody which selectively binds to a polypeptide of claim 5.

8. A method for detecting the presence of a PYRIN-9 polypeptide in a sample, comprising:
a) contacting the sample with a compound which selectively binds to a polypeptide of claim 5; and
b) determining whether the compound binds to the polypeptide in the sample.

9. A method for detecting the presence of a nucleic acid molecule of claim 1 in a sample, comprising the steps of:
a) contacting the sample with a nucleic acid probe or primer which selectively hybridizes to the nucleic acid molecule of claim 1; and
b) determining whether the nucleic acid probe or primer binds to a nucleic acid molecule in the sample.

10. A kit comprising a compound which selectively binds to a nucleic acid molecule of claim 1 and instructions for use.

11. A method for identifying a compound which binds to PYRIN-9, the method comprising the steps of:
a) contacting a polypeptide, or a cell expressing a polypeptide of claim 5 with a test compound; and
b) determining whether the polypeptide binds to the test compound.

12. The method of claim 11, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for PYRIN-9-mediated signal transduction;
d) detection of binding using an assay for proteolytic activity;
e) detection of binding to a pyrin domain; and
f) detection of binding to a CARD-5 polypeptide.

13. A method for identifying a compound which modulates the activity of PYRIN-9, comprising:
a) contacting a polypeptide, or a cell expressing a polypeptide of claim 5 with a test compound; and
b) determining the effect of the test compound on the activity of the polypeptide to thereby identify a compound which modulates the activity of the polypeptide.

14. The method of claim 13, wherein the polypeptide comprises the NBS domain of PYRIN-9 and the activity is binding of a nucleotide.

15. The method of claim 13, wherein the polypeptide comprises the NBS domain of PYRIN-9 and the activity is hydrolysis of a nucleotide.

16. The method of claim 13, wherein the activity is mediating cleavage of pro-IL-1β by caspase-1.

17. The method of claim 13, wherein the effect is determined by determining the phosphorylation state of PYRIN-9.

18. A method for modulating the activity of PYRIN-9, the method comprising contacting a polypeptide, or a cell expressing a polypeptide of claim 5, with a compound that binds to the polypeptide in a sufficient concentration to modulate the activity of the polypeptide.

19. A method for identifying a candidate compound which modulates the CARD-5-mediated activation of NF-κB, the method comprising:
a) measuring the binding of a first polypeptide comprising the pyrin domain of CARD-5 to a second polypeptide comprising the pyrin domain of PYRIN-9 in the presence of a test compound; and
b) comparing the binding of the first polypeptide to the second polypeptide measured in step (a) to the binding of the first polypeptide to the second polypeptide in the absence of the test compound,
wherein altered binding of the first polypeptide to the second polypeptide in the presence of the test compound compared the binding in the absence of the test compound indicates that the test compound is candidate compound for modulating the ASC-mediated activation of NF-κB.

20. A method for identifying a compound which modulates NF-κB activity, the method comprising:
a) providing a cell expressing a PYRIN-9 polypeptide of claim 5;
b) exposing the cell to a test compound; and
c) measuring the NF-κB activity of the cell in the presence of the test compound,
wherein altered activation in the presence of the test compound compared to the absence of the test compound indicates that the compound is a modulator of NF-kB activity.

21. The method of claim 20, wherein the cell additionally expresses a CARD-5 polypeptide.

22. The method of claim 20, wherein the compound binds to the pyrin domain of PYRIN-9.

23. The method of claim 20, wherein the compound binds to the NBS domain of PYRIN-9.

24. The method of claim 20, wherein the compound binds to the LRR domain of PYRIN-9.

25. A method for identifying a modulator of caspase-1 activity, the method comprising:
a) providing a cell expressing a PYRIN-9 polypeptide of claim 5;
b) exposing the cell to a test compound; and
c) measuring caspase-1 activity of the cell in the presence of the test compound,
wherein altered caspase-1 activity in the presence of the test compound compared to the absence of the test compound indicates that the compound is a modulator of caspase-1 activity.

26. The method of claim 25, wherein the cell additionally expresses a CARD-5 polypeptide.

27. A method of treating a disorder associated with inappropriate apoptosis in a subject by administering to the subject a compound which modulates the expression or activity of a PYRIN-9 polypeptide of claim 5 to thereby treat the apoptotic disorder.

28. A method for treating an inflammatory disorder in a subject by administering to the subject a compound which modulates the expression or activity of a PYRIN-9 polypeptide of claim 5 to thereby treat the inflammatory disorder.


SEQUENCE LISTING (5 sequences)

SEQ ID NO: 1 - DNA, 2679 bp, Homo sapiens
  CDS (1)...(2679)
atggaccagccagaggccccctgctccagcacggggccgcgcctcgcggtggcccgcgagctgctcctggctgcgctggaggaactgagccaagagcagctgaagcgcttccgccacaagctgcgcgacgtgggcccggacggacgcagcatcccgtgggggcggctggagcgcgcggacgccgtggacctcgcggagcagctggcccagttctacggcccggagcctgccctggaggtggcccgcaagaccctcaagagggcggacgcgcgcgacgtggcggcgcagctccaggagcggcggctgcagcggctcgggctcggctccgggacgctgctctccgtgtccgagtacaagaagaagtaccgggagcacgtgctgcagctgcacgctcgggtgaaggagaggaacgcccgctccgtgaagatcaccaagcgcttcaccaagctgctcatcgcgcccgagagcgccgccccggaggaggcgctggggcccgcggaagagcctgagccggggcgcgcgcggcgctcggacacgcacactttcaaccgcctcttccgccgcgacgaggagggccggcggccgctgaccgtggtgctgcagggcccggcgggcatcggcaagaccatggcggccaaaaagatcctgtacgactgggcggcgggcaagctgtaccagggccaggtggacttcgccttcttcatgccctgcggcgagctgctggagaggccgggcacgcgcagcctggctgacctgatcctggaccagtgccccgaccgcggcgcgccggtgccgcagatgctggcccagccgcagcggctgctcttcatcctggacggcgcggacgagctgccggcgctggggggccccgaggccgcgccctgcacagaccccttcgaggcggcgagcggcgcgcgggtgctaggcgggctgctgagcaaggcgctgctgcccacggccctcctgctggtgaccacgcgcgccgccgcccccgggaggctgcagggccgcctgtgttccccgcagtgcgccgaggtgcgcggcttctccgacaaggacaagaagaagtatttctacaagttcttccgggatgagaggagggccgagcgcgcctaccgcttcgtgaaggagaacgagacgctgttcgcgctgtgcttcgtgcccttcgtgtgctggatcgtgtgcaccgtgctgcgccagcagctggagctcggtcgggacctgtcgcgcacgtccaagaccaccacgtcagtgtacctgcttttcatcaccagcgttctgagctcggctccggtagccgacgggccccggttgcagggcgacctgcgcaatctgtgccgcctggcccgcgagggcgtcctcggacgcagggcgcagtttgccgagaaggaactggagcaactggagcttcgtggctccaaagtgcagacgctgtttctcagcaaaaaggagctgccgggcgtgctggagacagaggtcacctaccagttcatcgaccagagcttccaggagttcctcgcggcactgtcctacctgctggaggacggcggggtgcccaggaccgcggctggcggcgttgggacactcctgcgtggggacgcccagccgcacagccacttggtgctcaccacgcgcttcctcttcggactgctgagcgcggagcggatgcgcgacatcgagcgccacttcggctgcatggtttcagagcgtgtgaagcaggaggccctgcggtgggtgcagggacagggacagggctgccccggagtggcaccagaggtgaccgagggggccaaagggctcgaggacaccgaagagccagaggaggaggaggagggagaggagcccaactacccactggagttgctgtactgcctgtacgagacgcaggaggacgcgtttgtgcgccaagccctgtgccggttcccggagctggcgctgcagcgagtgcgcttctgccgcatggacgtggctgttctgagctactgcgtgaggtgctgccctgctggacaggcactgcggctgatcagctgcagattggttgctgcgcaggagaagaagaagaagagcctggggaagcggctccaggccagcctgggtggcggcagcagttctcaaggcaccacaaaacaactgccagcctcccttcttcatccactctttcaggcaatgactgacccactgtgccatctgagcagcctcacgctgtcccactgcaaactccctgacgcggtctgccgagacctttctgaggccctgagggcagcccccgcactgacggagctgggcctcctccacaacaggctcagtgaggcgggactgcgtatgctgagtgagggcctagcctggccgcagtgcagggtgcagacggtcagggtacagctgcctgacccccagcgagggctccagtacctggtgggtatgcttcggcagagccctgccctgaccaccctggatctcagcggctgccaactgcccgcccccatggtgacctacctgtgtgcagtcctgcagcaccagggatgcggcctgcagaccctcagtctggcctctgtggagctgagcgagcagtcactacaggagcttcaggctgtgaagagagcaaagccggatctggtcatcacacacccagcgctggacggccacccacaacctcccaaggaactcatctcgaccttctga

SEQ ID NO: 2 - protein, 892 aa, Homo sapiens
MDQPEAPCSSTGPRLAVARELLLAALEELSQEQLKRFRHKLRDVGPDGRSIPWGRLERADAVDLAEQLAQFYGPEPALEVARKTLKRADARDVAAQLQERRLQRLGLGSGTLLSVSEYKKKYREHVLQLHARVKERNARSVKITKRFTKLLIAPESAAPEEALGPAEEPEPGRARRSDTHTFNRLFRRDEEGRRPLTVVLQGPAGIGKTMAAKKILYDWAAGKLYQGQVDFAFFMPCGELLERPGTRSLADLILDQCPDRGAPVPQMLAQPQRLLFILDGADELPALGGPEAAPCTDPFEAASGARVLGGLLSKALLPTALLLVTTRAAAPGRLQGRLCSPQCAEVRGFSDKDKKKYFYKFFRDERRAERAYRFVKENETLFALCFVPFVCWIVCTVLRQQLELGRDLSRTSKTTTSVYLLFITSVLSSAPVADGPRLQGDLRNLCRLAREGVLGRRAQFAEKELEQLELRGSKVQTLFLSKKELPGVLETEVTYQFIDQSFQEFLAALSYLLEDGGVPRTAAGGVGTLLRGDAQPHSHLVLTTRFLFGLLSAERMRDIERHFGCMVSERVKQEALRWVQGQGQGCPGVAPEVTEGAKGLEDTEEPEEEEEGEEPNYPLELLYCLYETQEDAFVRQALCRFPELALQRVRFCRMDVAVLSYCVRCCPAGQALRLISCRLVAAQEKKKKSLGKRLQASLGGGSSSQGTTKQLPASLLHPLFQAMTDPLCHLSSLTLSHCKLPDAVCRDLSEALRAAPALTELGLLHNRLSEAGLRMLSEGLAWPQCRVQTVRVQLPDPQRGLQYLVGMLRQSPALTTLDLSGCQLPAPMVTYLCAVLQHQGCGLQTLSLASVELSEQSLQELQAVKRAKPDLVITHPALDGHPQPPKELISTF

SEQ ID NO: 3 - DNA, 585 bp, Homo sapiens
  CDS (1)...(585)
atggggcgcgcgcgcgacgccatcctggatgcgctggagaacctgaccgccgaggagctcaagaagttcaagctgaagctgctgtcggtgccgctgcgcgagggctacgggcgcatcccgcggggcgcgctgctgtccatggacgccttggacctcaccgacaagctggtcagcttctacctggagacctacggcgccgagctcaccgctaacgtgctgcgcgacatgggcctgcaggagatggccgggcagctgcaggcggccacgcaccagggctctggagccgcgccagctgggatccaggcccctcctcagtcggcagccaagccaggcctgcactttatagaccagcaccgggctgcgcttatcgcgagggtcacaaacgttgagtggctgctggatgctctgtacgggaaggtcctgacggatgagcagtaccaggcagtgcgggccgagcccaccaacccaagcaagatgcggaagctcttcagtttcacaccagcctggaactggacctgcaaggacttgctcctccaggccctaagggagtcccagtcctacctggtggaggacctggagcggagc

SEQ ID NO: 4 - protein, 195 aa, Homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 5 - protein, 77 aa, Homo sapiens
DAILDALENLTAEELKKFKLKLLSVPLREGYGRIPRGALLSMDALDLTDKLVSFYLETYGAELTANVLRDMGLQEMA